(19) United States
(12) Patent Application Publication
(10) Pub. No.: US 2026/0345736 A1
(43) Pub. Date: Nov. 5, 2026

(54) USE OF A LONG-ACTING OXYNTOMODULIN DERIVATIVE FOR TREAMENT OF FATTY LIVER DISEASE

(71) Applicant: Merck Sharp & Dohme LLC, Rahway, NJ (US)

(72) Inventors: Eirum CHAUDHRI, Old Tappan, NJ (US); Samuel ENGEL, New York, NY (US); Keith D. KAUFMAN, Westfield, NJ (US)

(73) Assignee: Merck Sharp & Dohme LLC, Rahway, NJ (US)

(21) Appl. No.: 19/491,164

(22) PCT Filed: Jun. 18, 2024
(86) PCT No.: PCT/US2024/034417

§ 371 Date: Dec. 8, 2025

Related U.S. Application Data
(60) Provisional application No. 63/509,894, filed on Jun. 23, 2023.

(51) Int. Cl. A61K 38/26; A61P 1/16

(52) U.S. Cl. CPC A61K 38/26; A61P 1/16

(57) ABSTRACT

The use of a long-acting oxyntomodulin derivative for treatment of fatty liver disease is disclosed. In particular, disclosed is the use of efinopegdutide and pharmaceutically acceptable salts thereof for treating nonalcoholic fatty liver disease (NAFLD) and nonalcoholic steatohepatitis (NASH).

### CROSS-REFERENCE TO RELATED APPLICATIONS

This application claims the benefit of U.S. Provisional Patent Application Ser. No. 63/509,894 filed Jun. 23, 2023, the entire contents of which are incorporated by reference herein.

### REFERENCE TO SEQUENCE LISTING SUBMITTED ELECTRONICALLY

The contents of the electronic sequence listing (25679-WO-PCT_SL.xml: Size: 7,539 bytes; and Date of Creation: Jul. 17, 2023) are herein incorporated by reference in their entirety.

### BACKGROUND OF THE INVENTION

### Field of the Invention

The present invention relates to the use of a long-acting oxyntomodulin derivative for treatment of fatty liver disease. In particular, the present invention relates to use of a long-acting oxyntomodulin derivative, e.g., efinopegdutide for treating nonalcoholic fatty liver disease and nonalcoholic steatohepatitis.

### Description of Related Art

Nonalcoholic fatty liver disease (NAFLD), a condition associated with increased accumulation of triglycerides in the liver, is estimated to affect approximately 25% of the global adult population.^{[0253]} NAFLD encompasses a broad spectrum of fatty liver disease, ranging from simple steatosis to nonalcoholic steatohepatitis (NASH), a form of fatty liver disease that is associated with chronic inflammation described histologically as steatohepatitis with or without fibrosis.^{[0254]} Approximately 20% of the NASH population will progress to cirrhosis, which is associated with increases in rates of hepatocellular carcinoma and all-cause and liver-related mortality.^{[0255]} NAFLD is increasingly being recognized as the hepatic manifestation of underlying metabolic dysregulation and is considered a consequence of obesity-related insulin resistance, resulting in increased trafficking of fatty acids from adipose tissue to the liver and de novo hepatic lipogenesis.^{[0256],[0257]} Being overweight and obesity are considered the primary pathological drivers of metabolic disease, NAFLD and, by extension, NASH.^{[0257]}

Currently, there are no approved therapies for the treatment of NASH. Management of NAFLD/NASH is focused on lifestyle modification such as diet and exercise, directed mainly at weight loss. Pioglitazone and high-dose vitamin E (800 LU/day) have been recommended as pharmacotherapies for biopsy-proven NASH, with pioglitazone recommended in those with type 2 diabetes mellitus (T2DM) and Vitamin E recommended in those without diabetes.^{[0258]}

Glucagon-like peptide-1 (GLP-1) agonism is associated with reductions in serum glucose and weight loss. GLP-1 receptor agonists enhance glucose-stimulated insulin secretion and have become useful treatments for Type 2 Diabetes Mellitus (T2DM). At doses that have been developed for diabetes indications (e.g., liraglutide dose up to 1.8 mg daily, semaglutide dose up to 2 mg weekly), GLP-1 receptor agonists are associated with weight loss of approximately 3% to 5%, generally attributed to reductions in food intake. More recently, higher dose administration of GLP-1 receptor agonists has been pursued for weight loss indications. At the approved doses for weight loss, liraglutide administered at a dose of 3 mg subcutaneously (SC) daily over 56 weeks resulted in weight loss of approximately 7.4%,^{[0259]} while semaglutide at a dose of 2.4 mg SC once weekly over 68 weeks resulted in approximately 15% weight loss.^{[0260]} The weight loss associated with GLP-1 agonists has been shown to be associated with decreased hepatic inflammation in patients with NASH. The Liraglutide Efficacy and Action in NASH (LEAN) Phase 2 study showed that 39% (9/23) of participants who received liraglutide at a dose of 1.8 mg SC daily and who underwent an end-of-treatment liver biopsy after 48 weeks had resolution of NASH compared with 9% (2/22) in the placebo group.^{[0261]} A Phase 2b study with semaglutide at doses of 0.1 mg, 0.2 mg, or 0.4 mg SC once daily (total weekly dose of 0.7 mg to 2.8 mg) showed histologic resolution of NASH without worsening of fibrosis in 40.4% to 58.9% of participants compared to placebo (17.2%) after 72 weeks of dosing, albeit without significant improvement in fibrosis.^{[262]}

Glucagon receptor activation has a number of effects that may complement the beneficial effects of GLP-1 receptor agonism for the treatment of NASH.^{[0263]} Glucagon has been shown to induce weight loss by reducing food intake and increasing energy expenditure. In addition to reducing liver fat content (LFC) indirectly through weight loss, glucagon agonism may also reduce LFC by acting on the liver directly to stimulate fatty acid oxidation and reduce lipogenesis.^{[0264]}

Oxyntomodulin, a 37 amino acid peptide product of the proglucagon gene released from L-cells of the small intestine in response to food ingestion, has been shown to decrease appetite and body weight in overweight and obese individuals.^{[0265],[0266]} Efinopegdutide is along-acting GLP-1 receptor/glucagon receptor dual agonist containing an oxyntomodulin derivative conjugated to the Fc region of human IgG4 and displaying a GLP-1 receptor to glucagon receptor relative potency of approximately 2:1. The effects of efinopegdutide on weight loss has been studied in two Phase 2 dose-ranging studies in obese patients with and without T2DM.^{[0267],[0268]} In both studies, treatment with efinopegdutide resulted in a significant, dose-dependent reduction in body weight.^{[0267],[0268]}

### BRIEF SUMMARY OF THE INVENTION

The present invention provides a treatment for fatty liver diseases, including nonalcoholic fatty liver disease (NAFLD) and nonalcoholic steatohepatitis (NASH) using the long-acting GLP-1 receptor/glucagon receptor dual agonist efinopegdutide. The present invention is predicated on the discovery from a clinical trial comprising patients with elevated liver fat concentration (LFC) (a mean LFC of 20.3%) that administering efinopegdutide to these patients at a dose of 10 mg once weekly for about 24 weeks resulted in a reduction of LFC in these patients. The mean LFC in the patient group at week 24 was about 4.6%, which is within the “normal” range of less than 5%.

In an embodiment of the present invention, a method for reducing LFC in an individual with fatty liver disease, comprises administering from about 2 mg to about 10 mg (and in particular embodiments, about 2 mg, 4 mg, 7 mg, or about 10 mg) of efinopegdutide or a pharmaceutically acceptable salt thereof to the individual with fatty liver disease once weekly for a time sufficient to reduce the LFC in the individual with fatty liver disease to less than 5%. The LFC may be assessed by magnetic resonance imaging-estimated proton density fat fraction (MRI-PDFF). The efinopegdutide or pharmaceutically acceptable salt thereof may be administered subcutaneously.

In a further embodiment of the method, the time sufficient to reduce the LFC to less than 5% may be about 24 weeks or more. In specific embodiments, the time sufficient to reduce the LFC to less than 5% may be However, depending on the LFC of the individual, the time sufficient to reduce the LFC in the individual may be more than 24 weeks or may be less than 24 weeks. In specific embodiments, the time sufficient to reduce the LFC in the individual may be more than 24 weeks but less than 52 weeks. It is to be understood that the time sufficient to reduce the LFC to less than 5% in any particular individual is dependent on the nature of the disease in the individual and the individual's response to treatment.

In a further embodiment of the method, the fatty liver disease is nonalcoholic fatty liver disease (NAFLD) or nonalcoholic steatohepatitis (NASH). In a further embodiment of the method, the NASH is pre-cirrhotic NASH or cirrhotic NASH. Depending on the LFC of the individual and the severity of the fatty liver disease, the time sufficient to reduce the LFC in the individual to less than 5% may be more than 24 weeks or may be less than 24 weeks.

In a further embodiment of the method, the individual with fatty liver disease has an LFC of 5% or greater prior to administering the 10 mg of efinopegdutide or pharmaceutically acceptable salt thereof. In a further embodiment of the method, the individual with fatty liver disease has an LFC of 10% or greater prior to administering the 10 mg of efinopegdutide or pharmaceutically acceptable salt thereof. In a further embodiment of the method, the individual with fatty liver disease has an LFC of 15% or greater prior to administering the 10 mg of efinopegdutide or pharmaceutically acceptable salt thereof. In a further embodiment of the method, the individual with fatty liver disease has an LFC of 20% or greater prior to administering the 10 mg of efinopegdutide or pharmaceutically acceptable salt thereof.

In a further embodiment of the method, the individual has type 1 diabetes mellitus (T1DM) or type 2 diabetes mellitus (T2M). In a further embodiment of the method, the individual has type 2 diabetes mellitus (T2M) and is overweight or obese. In a further embodiment of the method, the individual is overweight or obese.

The present invention further provides a method for reducing liver fat content (LFC) to less than 5% in an individual with elevated LFC comprising in the following order the steps of (a) administering to the individual about 2.0 mg or about 2.4 mg doses of efinopegdutide or pharmaceutically acceptable salt thereof once a week for four weeks; (b) administering to the individual about 4.0 mg or about 5.0 mg doses of efinopegdutide or pharmaceutically acceptable salt thereof once a week for four weeks; and, (c) administering to the individual about 10 mg doses of efinopegdutide or pharmaceutically acceptable salt thereof once weekly for a time sufficient to reduce the LFC to less than 5%. The LFC may be assessed by MRI-PDFF. The efinopegdutide or pharmaceutically acceptable salt thereof may be administered subcutaneously.

In a further embodiment of the method, the time sufficient to reduce the liver fat to less than 5% is about 24 weeks. However, depending on the LFC of the individual, the time sufficient to reduce the LFC in the individual to less than 5% may be more than 24 weeks or may be less than 24 weeks. In a further embodiment of the method, the individual with elevated LFC has a fatty liver disease. In particular embodiments, the fatty liver disease is nonalcoholic fatty liver disease (NAFLD). In a further embodiment, the fatty liver disease is nonalcoholic steatohepatitis (NASH). In a further embodiment of the method, the NASH is pre-cirrhotic NASH or cirrhotic NASH. Depending on the LFC of the individual and the severity of the fatty liver disease, the time sufficient to reduce the LFC in the individual to less than 5% may be more than 24 weeks or may be less than 24 weeks.

In a further embodiment of the method, the individual with fatty liver disease has an LFC of 5% or greater prior to beginning the method. In a further embodiment of the method, the individual with fatty liver disease has an LFC of 10% or greater prior to beginning the method. In a further embodiment of the method, the individual with fatty liver disease has an LFC of 15% or greater prior to beginning the method. In a further embodiment of the method, the individual with fatty liver disease has an LFC of 20% or greater prior to beginning the method.

In a further embodiment of the method, the individual has type 1 diabetes mellitus (T1DM) or type 2 diabetes mellitus (T2M). In a further embodiment of the method, the individual has type 2 diabetes mellitus (T2M) and is overweight or obese. In a further embodiment of the method, the individual is overweight or obese.

The present invention further provides for use of efinopegdutide or pharmaceutically acceptable salt thereof for the manufacture of a medicament for reducing liver fat content (LFC) in individuals with elevated LFC to a LFC of less than 5%. In a further embodiment, the treatment comprises from about 2 mg to about 10 mg (and in particular embodiments, about 2 mg, 2.4 mg, 4 mg, 7 mg, or about 10 mg) of efinopegdutide or a pharmaceutically acceptable salt thereof.

The present invention further provides efinopegdutide or a pharmaceutically acceptable salt thereof for reducing liver fat content (LFC) in individuals with elevated LFC to a LFC of less than 5%. In a further embodiment, the efinopegdutide or pharmaceutically acceptable salt thereof is provided in doses from about 2 mg to about 10 mg (and in particular embodiments, about 2 mg, 2.4 mg, 4 mg, 7 mg, or about 10 mg).

### BRIEF DESCRIPTION OF THE DRAWINGS

FIG. 1 shows the study design.

FIG. 2 shows the disposition of participants.

FIG. 3A shows the least squares (LS) mean relative reduction from baseline at Week 24 in liver fat content (LFC).

FIG. 3B shows proportions of participants with relative reductions from baseline in LFC at Week 24 of ≥30%, ≥50% and ≥70%.

FIG. 3C shows mean relative reductions from baseline in LFC at Week 24 by weight-loss category (≤5%, >5% to ≤10%, and >10% reduction in body weight from baseline).

FIG. 4A shows the mean (SE) change from baseline over time in heart rate.

FIG. 4B shows the mean (SE) change from baseline over time in systolic blood pressure, and (FIG. 4C) diastolic blood pressure.

FIG. 5A shows the mean (SE) change from baseline over time in alanine aminotransferase (ALT).

FIG. 5B shows the mean (SE) change from baseline over time in aspartate aminotransferase (AST).

FIG. 6A and FIG. 6B together show the structure of efinopegdutide. Efinopegdutide comprises an oxyntomodulin derivative having the amino acid sequence set forth in SEQ ID NO: 2 conjugated to the N-terminus of one Fc of an aglycosylated IgG4 Fc homodimer, wherein the aglycosylated IgG4 Fc has the amino acid sequence set forth in SEQ ID NO: 3. The molecular weight of efinopegdutide is about 64 kDa.

### DETAILED DESCRIPTION OF THE INVENTION

### DEFINITIONS

As used herein, the term “about” refers to plus or minus up to 10% of the value it modifies (rounded up to the nearest whole number if the value is not sub-dividable, such as a number of molecules or nucleotides). The term “about”, when modifying the quantity (e.g., mg) of a substance or composition, a parameter of a substance or composition or a parameter used in characterizing a step in a method, or the like, can account for the variation in the numerical quantity that can occur. For example, such variation can occur through typical measuring, handling and sampling procedures involved in the preparation, characterization and/or use of the substance or composition, through inadvertent error in these procedures, through differences in the manufacture, source, or purity of the ingredients employed to make or use the compositions or carry out the procedures.

As used herein, the term “oxyntomodulin” refers to a peptide produced from pre-glucagon that a precursor of glucagon. In the present invention, oxyntomodulin is meant to include native oxyntomodulin and its precursor, analog (derivative), fragment and variant. Oxyntomodulin has an amino acid sequence set forth in SEQ ID NO: 1.

As used herein, the term “oxyntomodulin variant” is a peptide that has one or more amino acid residues different from those of the amino acid sequence of native oxyntomodulin and possesses a function of activating both GLP-1 and glucagon receptors. The oxyntomodulin variant can be prepared by any one of substitution, addition, deletion, modification, or a combination thereof of some amino acids of native oxyntomodulin.

As used herein, the term “oxyntomodulin derivative” refers to a peptide, peptide derivative or peptide mimic that is prepared by the addition, deletion or substitution of some amino acids of native oxyntomodulin and can activate both the GLP-1 receptor and glucagon receptor at a high level compared to the level activated by native oxyntomodulin. Efinopegdutide is a long-acting oxyntomodulin derivative.

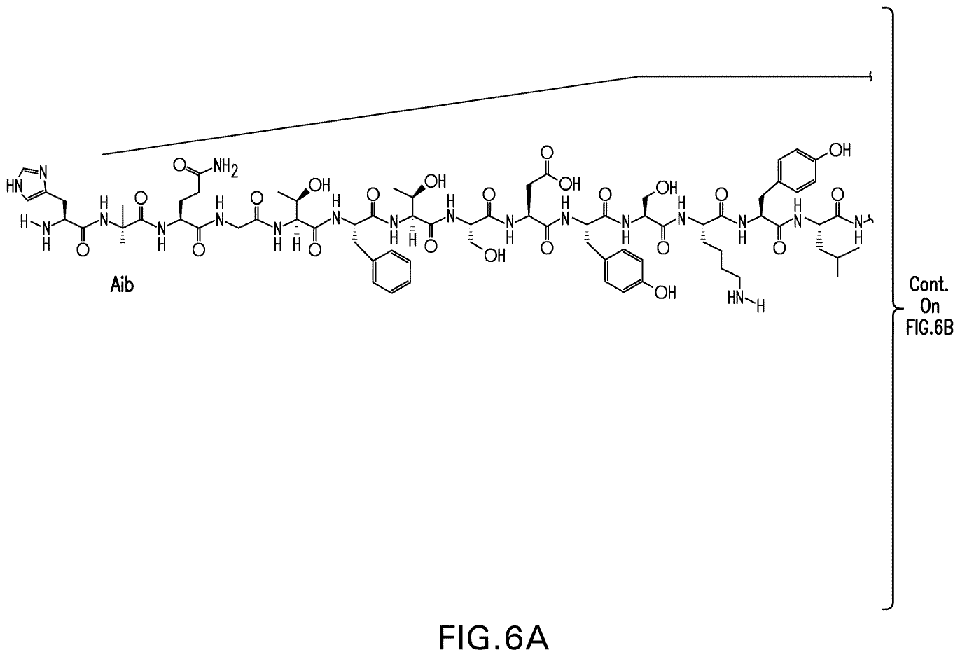

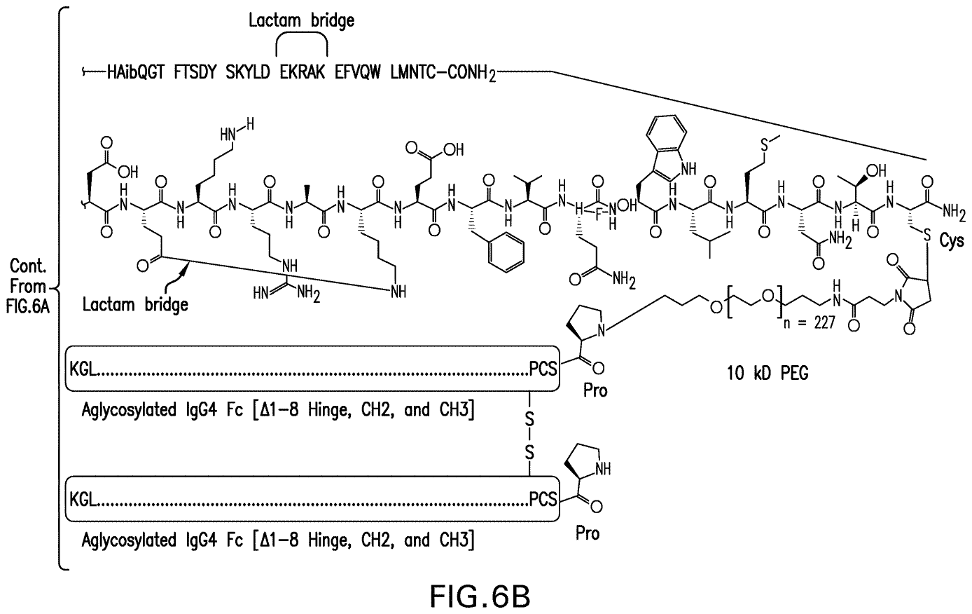

As used herein, the term “efinopegdutide” refers to a biologic compound comprising an oxyntomodulin derivative having the amino acid sequence set forth in SEQ ID NO: 2 conjugated at the C-terminal cysteine residue thereof to the N-terminus of an aglycosylated IgG4 Fc analog having a deletion of amino acids 1-8 of the hinge region (SEQ ID NO: 3) by a non-peptide 10 kD polyethylene linker having the formula shown in FIG. 6A/FIG. 6B. Efinopegdutide has a molecular weight of about 64 kDa: a 10 mg dose of efinopegdutide is about 150 nanomoles. Efinopegdutide may in particular embodiments the aglycosylated IgG4 Fc analog may lack a C-terminal lysine (See SEQ ID NO: 4).

As used herein, the term “semaglutide” refers to a compound comprising a GLP-1 derivative having the amino acid sequence set forth in SEQ ID NO: 4 conjugated at the lysine at position 20 thereof to a polyethylene linker linked to a C18 fatty diacid. The molecular weight of semaglutide is 4113.58 Da: a 1 mg dose of semaglutide is about 2.4 nanomoles.

As used herein, the term “fatty liver disease” or “hepatic steatosis” refers to a condition in which the ratio of fats in the weight of the liver is 5% or more. In the present invention, fatty liver diseases include non-alcoholic fatty liver disease (NAFLD), alcoholic fatty liver disease (AFLD), nutritional fatty liver disease (NFLD), starvation fatty liver disease (SFLD), obesity fatty liver disease, diabetic fatty liver disease or steatohepatitis. The NAFLD is meant to include primary and secondary NAFLD, but may also be a NAFLD resulting from primary hyperlipidemia, diabetes or obesity.

As used herein, the term “non-alcoholic fatty liver disease” or “NAFLD” refers to fatty liver cases in which there is no history of alcohol consumption or in which alcohol consumption is not related to the occurrence. The fatty liver refers to a phenomenon in which there is abnormal accumulation of triglyceride in liver cells, compared to normal levels of triglyceride. About 5% of normal liver consists of fat tissue and the main components of the fat are triglycerides, fatty acids, phospholipids, cholesterols, and cholesterol esters. However, once the fatty liver occurs, most of the components are replaced with triglyceride. If the amount of triglycerides is more than 5% of the liver weight, it is diagnosed as fatty liver. The fatty liver is caused by a lipid metabolism disorder or a defect in the process of carrying excessive fat in the liver cells, and is mainly caused by disorders of lipid metabolism in the liver. Most of the fat accumulated in the fatty liver may be a triglyceride. The NAFLD includes non-alcoholic fatty liver (NAFL), non-alcoholic steatohepatitis (NASH), cirrhosis, liver cancer, and the like, but the fatty liver disease to be prevented or treated with the composition of the present invention is included without limitation. In addition, in the present invention, NAFLD is meant to include simple steatosis, NASH, liver fibrosis and/or liver cirrhosis which result from the progression of such diseases.

An individual having NAFLD has elevated LFC and may further have a body mass index (BMI) 25 kg/m² or greater and 50 kg/m² or less. The individual may further have no history of T2DM or a history of T2DM with an A1C 8.5% or less.

As used herein the term “steatosis” or “fatty change” of fatty liver is the accumulation of abnormal amounts of lipids in 5% or more hepatic cells.

As used herein, the term “elevated liver fat content” or “elevated LFC” refers to an LFC in an individual of 10% or more as may be assessed by magnetic resonance imaging-estimated proton density fat fraction (MRI-PDFF). Histologically, the liver is considered steatotic when 5% or more of hepatocytes in a tissue section stained with hematoxylin and eosin contain macrovesicular steatosis.

As used herein, the term “hyperlipidemia” refers to a condition associated with abnormally elevated levels of lipids, such as free cholesterol, cholesterol esters, phospholipids and triglycerides, in blood. Although hyperlipidemia does not show specific symptoms by itself, excessive lipids in blood adhere to the blood vessel walls to reduce the blood vessel size and cause atherosclerosis by inflammatory reactions. For this reason, coronary heart disease, cerebrovascular disease, obstruction of peripheral blood vessels, etc., can occur.

As used herein, the term “obesity” refers to a body-mass index (BMI) of greater than or equal to 30 kg/m². Obesity generally results from an energy imbalance in which energy intake exceeds energy expenditure over time. Obesity is a metabolic disease that affects the entire body and highly likely to lead to diabetes and hyperlipidemia. In addition, obesity is related to sexual dysfunction, arthritis, and an increased risk of the development of cardiovascular diseases, and is also related to the development of fatty liver disease and cancer in some cases.

As used herein, the term “overweight” means, with regard to an individual, one having a BMI of greater than or equal to 25 kg/m² but less than 30 kg/m².

As used herein, the term “diabetes” or “diabetes mellitus” refers to a kind of metabolic disease in which insulin secretion is insufficient or normal functions are not made. Diabetes is characterized by hyperglycemia, which is an increased blood glucose level, thereby causing various symptoms, and thus, glucose is excreted with urine. Type 1 diabetes mellitus (T1DM), once known as juvenile diabetes or insulin-dependent diabetes, is a chronic condition. In this condition, the pancreas makes little or no insulin. In type 2 diabetes mellitus (T2DM), once known as adult-onset diabetes but which may also occur in children, the pancreas makes less insulin than used to, and the body becomes resistant to insulin. The main difference between T1DM and T2DM is that T1DM is a genetic condition that often shows up early in life, and T2DM is mainly lifestyle-related and develops over time.

As used herein, the term “prevention” refers to all actions that inhibit or delay the development of a target disease. As used herein, the term “prevention” means administering efinopegdutide to an individual in a therapeutically effective amount for a time sufficient to inhibit or delay the development of fatty liver disease, such as NAFLD or NASH, or the progression of fatty liver disease from one stage to a more advanced stage. For example, to inhibit or delay the progression of early stages of NAFLD to the more advanced form non-alcoholic steatohepatitis (NASH), and ultimately to cirrhosis or liver cancer.

As used herein, the term “treatment”. “treat”, or “treating” refers to administering a therapeutic agent, to a subject or patient having a disease or disorder, or being suspected of having a disease or disorder, for which the agent has therapeutic activity or prophylactic activity. For example, administering efinopegdutide to an individual in a therapeutically effective amount for a time sufficient to alleviate, ameliorate, or relieve fatty liver disease, for example, NAFLD or NASH, or to delay progression of one stage of fatty liver disease to a more advanced stage of fatty liver disease. Use of efinopegdutide may provide both therapeutic activity and prophylactic activity. Used therapeutically in an individual with fatty liver disease, a therapeutically effective amount of efinopegdutide will over time alleviate, ameliorate, or relieve the symptoms of the fatty liver disease. Used prophylactically in an individual with fatty liver disease, a therapeutically effective amount of efinopegdutide will over time inhibit or delay the progression of the fatty liver disease to a more advanced stage, e.g., the progression from NAFLD to NASH, to cirrhosis, or liver cancer.

As used herein. “therapeutically effective amount” refers to an amount, concentration, or dose of efinopegdutide, or a pharmaceutically acceptable salt thereof, which upon single or multiple dose administration to an individual in need thereof, provides a desired effect in such an individual under diagnosis or treatment (i.e., may produce a clinically measurable difference in a condition of the individual such as, for example, a reduction in LFC). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular incretin analog administered, the mode of administration, the bioavailability characteristics of the preparation administered, the dose regimen selected, the use of concomitant medication, and other relevant circumstances. For example, a therapeutically effective amount of efinopegdutide may be 10 mg. As shown herein, when 10 mg of efinopegdutide is administered to an individual with an elevated liver fat content (LFC) once weekly for a period of 24 weeks, the efinopegdutide effected a reduction of LFC in the individual to less than 5%.

As used herein, “pharmaceutically acceptable salt” is well known to one of skill in the art. Pharmaceutically acceptable salts and common techniques for preparing them are well known in the art (see, e.g., Stahl et al., Handbook of Pharmaceutical Salts: Properties, Selection and Use, 2nd Revised Edition (Wiley-VCH, 2011)). Pharmaceutically acceptable salts for use herein include, but are not limited to, sodium, trifluoroacetate, hydrochloride, citrate, and/or acetate salts.

### Introduction

Non-alcoholic fatty liver disease (NAFLD) is a clinicopathologic entity increasingly recognized as a major health burden in developed countries. It includes a spectrum of liver damage ranging from simple steatosis to nonalcoholic steatohepatitis (NASH), advanced fibrosis, and rarely, progression to cirrhosis. NAFLD is strongly associated to the features of metabolic syndrome and that encompasses nonalcoholic fatty liver (NAFL), nonalcoholic steatohepatitis (NASH), liver cirrhosis, and hepatocellular carcinomas. The occurrence of NAFLDs appears to correlate with the increase in obesity and diabetes. In the U.S., NAFLD affects between 80 and 100 million individuals, among whom nearly 25% advance to NASH. NASH is one of the most potent reasons for liver transplantation. The current annual medical and societal costs of NAFLD are estimated at $292 billion in the United States. Obesity is a common indication of the presence of NAFLD and NAFLD can progress to NASH, a more aggressive form of disease. Currently, there are no approved therapies for NASH.

Non-alcoholic fatty liver disease is known to be caused by various etiologies such as insulin resistance, lipotoxicity, and inflammatory responses. Among these, insulin resistance is believed to be a key pathogenic factor in both NAFLD and metabolic syndrome. Significant effort has been made to improve the insulin resistance to prevent/treat non-alcoholic fatty liver disease. For example, clinical trials for thiazolidnedinones (TZD) or metformin, a kind of insulin sensitizer, have been actively conducted (see, Hepatology (2003) 38: 1008-17. J Clin Invest (2001) 108: 1167-74).

However, in the case of treatment with the TZD-based drugs, there are the disadvantages of a large weight gain and slow fluid flow, and thus, the use of such treatments may not be suitable for therapies intended for patients with a heart disease. In addition to the TZD-based drugs, clinical tests using GLP-1 receptor agonists such as Victoza® or Byetta® for nonalcoholic fatty liver disease have been conducted. However, for these GLP-1 receptor agonists, the in vivo half-life is extremely short, and thus patients must undergo repeated administrations at least once or even twice per day, which in turn makes them inconvenient for patients to use and unsuitable for long-term therapies. Furthermore, simply using general therapeutic agents designed for treating diabetes as a therapeutic agent for NAFLD through the mechanism of improving insulin resistance has disadvantages such as inducing various side-effects. Therefore, whether a drug known to be effective in the treatment of diabetes, such as a drug for improving insulin resistance, can definitely be used as a therapeutic agent for NAFLD, remains controversial. Thus, there remains a need to develop drugs capable of treating NAFLD while securing patient's convenience with minimal or no side-effects.

Efinopegdutide has been disclosed in U.S. Pat. No. 9,731,031 and the oxyntomodulin derivative comprising efinopegdutide has been disclosed in U.S. Pat. No. 9,527,898. The use of efinopegdutide for treating diabetes or obesity has been disclosed in U.S. patent Ser. No. 10/550,168, in combination with long-acting insulin for treating diabetes has been disclosed in U.S. patent Ser. No. 10/188,703, for treating severe diabetes has been disclosed in International PCT Publication WO2019171352, and for treating severe non-diabetic obesity has been disclosed in International PCT Publication WO2020128967. The use of efinopegdutide for treating liver diseases such as NAFLD or NASH has been disclosed in U.S. Pat. No. 9,901,621 and U.S. patent Ser. No. 10/233,230.

Efinopegdutide has the subject of nine clinical trials for studying the safety and efficacy of efinopegdutide in non-diabetic severely obese patients (NCT03486392), severely obese T2DM patients (NCT03586830), T2DM patients (NCT03235219), healthy overweight/obese patients (NCT03586843), patients with varying degrees of renal failure (NCT03546205). Two clinical trials related to safety, tolerability, pharmacokinetics, and pharmacodynamics were terminated (NCT02862431 and NCT03618160). Two other trials include the effect of efinopegdutide on cardiac repolarization (NCT003606057) and its use in patients with NAFLD compared to semaglutide (NCT04944992).

### Use of Efinopegdutide for Treating Fatty Liver Disease

The present invention provides a use of efinopegdutide or pharmaceutically acceptable salt thereof at a dose of 10 mg once weekly for a time sufficient to reduce the liver fat content (LFC) in an individual that has elevated LFC to a LFC of less than 5%.

The present invention provides a dosage regimen for reducing LFC to less than 5% in an individual with elevated LFC comprising in the following order the steps of (a) administering to the individual 2.4 mg doses of efinopegdutide or pharmaceutically acceptable salt thereof once a week for four weeks, (b) administering to the individual 5.0 mg doses of efinopegdutide or pharmaceutically acceptable salt thereof once a week for four weeks, and (c) administering to the individual 10 mg doses of efinopegdutide or pharmaceutically acceptable salt thereof once weekly for a time sufficient to reduce the LFC to less than 5%. Depending on the LFC of the individual, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for maintaining the liver fat at less than 5% in individuals that have undergone a reduction of LFC comprising administering to the individual 10 mg doses of efinopegdutide or pharmaceutically acceptable salt thereof on a once weekly basis.

In a particular embodiment, the present invention further provides a method for treating fatty liver disease in an individual by administering to the individual efinopegdutide or pharmaceutically acceptable salt thereof in once weekly doses of 10 mg for a time sufficient to reduce the LFC of the individual to less than 5% thereby treating the fatty liver disease of the individual. The present invention further provides a dosage regimen for treating an individual with fatty liver disease comprising in the following order the steps of (a) administering to the individual doses of 2.4 mg efinopegdutide or pharmaceutically acceptable salt thereof once a week for four weeks, (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating the fatty liver disease of the individual.

Depending on the LFC of the individual and the severity of the fatty liver disease, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for fatty liver disease comprising efinopegdutide or pharmaceutically acceptable salt thereof in an amount and for a time to reduce the LFC of the individual to less than 5%, wherein the maintenance therapy comprises administering to the individual 10 mg of efinopegdutide or pharmaceutically acceptable salt thereof on a once weekly basis.

In a particular embodiment, the present invention further provides a method for treating NAFLD in an individual by administering to the individual efinopegdutide or pharmaceutically acceptable salt thereof in once weekly doses of 10 mg for a time sufficient to reduce the LFC of the individual to less than 5% thereby treating the NAFLD of the individual. The present invention further provides a dosage regimen for treating an individual with NAFLD comprising in the following order the steps of (a) administering to the individual doses of 2.4 mg efinopegdutide or pharmaceutically acceptable salt thereof once a week for four weeks, (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating the NAFLD of the individual.

Depending on the LFC of the individual and the severity of the NAFLD, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for NAFLD comprising efinopegdutide or pharmaceutically acceptable salt thereof in an amount and for a time to reduce the LFC of the individual to less than 5%, wherein the maintenance therapy comprises administering to the individual 10 mg of efinopegdutide or pharmaceutically acceptable salt thereof on a once weekly basis.

In a particular embodiment, the present invention further provides a method for treating NASH in an individual by administering to the individual efinopegdutide or pharmaceutically acceptable salt thereof in once weekly doses of 10 mg for a time sufficient to reduce the LFC of the individual to less than 5% thereby treating the NASH of the individual. The present invention further provides a dosage regimen for treating an individual with NASH comprising in the following order the steps of (a) administering to the individual 2.4 mg doses of efinopegdutide or pharmaceutically acceptable salt thereof once a week for four weeks, (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating the NASH of the individual.

Depending on the LFC of the individual and the severity of the NASH, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for NASH comprising efinopegdutide or pharmaceutically acceptable salt thereof in an amount and for a time to reduce the LFC of the individual to less than 5%, wherein the maintenance therapy comprises administering to the individual 10 mg doses of efinopegdutide or pharmaceutically acceptable salt thereof on a once weekly basis.

In a particular embodiment, the present invention further provides a method for treating pre-cirrhotic NASH in an individual by administering to the individual efinopegdutide or pharmaceutically acceptable salt thereof in once weekly doses of 10 mg for a time sufficient to reduce the LFC of the individual to less than 5% thereby treating the pre-cirrhotic NASH of the individual. The present invention further provides a dosage regimen for treating an individual with pre-cirrhotic NASH comprising in the following order the steps of (a) administering to the individual 2.4 mg doses of efinopegdutide or pharmaceutically acceptable salt thereof once a week for four weeks, (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating the pre-cirrhotic NASH of the individual.

Depending on the LFC of the individual and the severity of the pre-cirrhotic NASH, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for pre-cirrhotic NASH comprising efinopegdutide or pharmaceutically acceptable salt thereof to the individual in an amount and for a time to reduce the LFC of the individual to less than 5%, wherein the maintenance therapy comprises administering to the individual 10 mg doses of efinopegdutide or pharmaceutically acceptable salt thereof on a once weekly basis.

In a particular embodiment, the present invention further provides a method for treating cirrhotic NASH in an individual by administering to the individual efinopegdutide or pharmaceutically acceptable salt thereof in once weekly doses of 10 mg for a time sufficient to reduce the LFC of the individual to less than 5% thereby treating the cirrhotic NASH of the individual. The present invention further provides a dosage regimen for treating an individual with cirrhotic NASH comprising in the following order the steps of (a) administering to the individual 2.4 mg doses of efinopegdutide or pharmaceutically acceptable salt thereof once a week for four weeks. (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating the cirrhotic NASH of the individual.

Depending on the LFC of the individual and the severity of the cirrhotic NASH, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for cirrhotic NASH comprising efinopegdutide or pharmaceutically acceptable salt thereof in an amount and for a time to reduce the LFC of the individual to less than 5% wherein the maintenance therapy comprises administering to the individual 10 mg doses of efinopegdutide or pharmaceutically acceptable salt thereof on a once weekly basis.

In a particular embodiment, the present invention further provides a method for treating or inhibiting steatosis in an individual by administering to the individual efinopegdutide or pharmaceutically acceptable salt thereof in once weekly doses of 10 mg for a time sufficient to reduce the LFC of the individual to less than 5% thereby treating or inhibiting the steatosis. The present invention further provides a dosage regimen for treating or inhibiting steatosis in an individual comprising in the following order the steps of (a) administering to the individual 2.4 mg doses of efinopegdutide or pharmaceutically acceptable salt thereof once a week for four weeks, (b) administering to the individual 5.0 mg doses of efinopegdutide or pharmaceutically acceptable salt thereof once a week for four weeks, and administering to the individual 10 mg doses of efinopegdutide or pharmaceutically acceptable salt thereof once weekly for a time sufficient to reduce the LFC to less than 5% thereby treating or inhibiting the steatosis.

Depending on the LFC of the individual and the severity of the steatosis, the time sufficient to reduce the LFC in the individual to less than 5% may be more than 24 weeks or may be less than 24 weeks. In particular embodiments, the efinopegdutide or pharmaceutically acceptable salt thereof is administered at 10 mg once weekly for at least 10 weeks, at least 20 weeks, or at least 24 weeks to effect a reduction of LFC to less than 5% in the individual.

The present invention further provides a maintenance therapy for an individual who has undergone a treatment therapy for steatosis comprising efinopegdutide or pharmaceutically acceptable salt thereof in an amount and for a time to reduce the LFC of the individual to less than 5% wherein the maintenance therapy comprises administering to the individual 10 mg doses of efinopegdutide or pharmaceutically acceptable salt thereof on a once weekly basis.

The utility of the 10 mg once weekly dose for reducing LFC in an individual to less than 5% was discovered by the inventors while performing a Phase 2b study comparing the efficacy and safety of GLP-1 and glucagon receptor co-agonism provided by efinopegdutide at a 10 mg dose to GLP-1 agonism alone provided by semaglutide at a 1 mg dose in patients with NAFLD (see the Examples). In the study, both active treatments produced clinically meaningful reductions from baseline in LFC. However, efinopegdutide provided a significantly greater reduction in LFC than semaglutide. Treatment with efinopegdutide allowed two-thirds of participants to achieve anormal LFC level (less than 5%) compared with less than one-fifth of participants who were on semaglutide. A 10 mg dose of efinopegdutide (MW 64 kDa) is about 1.5 nanomoles and a 1 mg dose of semaglutide (MW 6113.58 Da) is about 2.4 nanomoles.

The significantly larger reduction in LFC with efinopegdutide than with semaglutide occurred in spite of both treatments producing similar reductions in body weight. This study was initiated prior to the availability of the higher dose (2.4 mg weekly) of semaglutide that is currently approved for the treatment of obesity and that is being evaluated in a Phase 3 study as a potential therapeutic option for patients with NASH. While the greater magnitude of weight loss observed with higher doses of semaglutide may provide some additional antisteatotic efficacy compared to the dose used in this study, prior studies with higher doses of semaglutide have resulted in relative reductions of liver fat of 36% to 46% at 6 months.^{[0270]-[0272]}

Consistent with the experience with dietary^{[12731]}, pharmacologic^{[0274]}, and surgical therapies^{[0275],[0276]} for weight loss,participants in both treatment groups with larger body weight reductions had greater reductions in LFC. The relative reductions from baseline in LFC at Week 24 by specific weight-loss category (≤5%, >5% to ≤10%, and >10% reduction in body weight from baseline) were all greater in the efinopegdutide group than in the semaglutide group. Of particular note was the magnitude of LFC reduction in participants in the lowest weight-loss category. Among those with 5% or less weight loss from baseline, the reduction in LFC with efinopegdutide was 52.4% compared to a reduction of 13.4% in the semaglutide group. These findings suggest that other mechanisms, beyond those related to weight loss, contributed to the greater LFC-reducing effect with efinopegdutide compared to semaglutide. This difference may be due to the glucagon receptor agonism of efinopegdutide directly stimulating fatty acid oxidation and reducing lipogenesis in the liver.^{[0264]}

The study showed that treatment with efinopegdutide was generally well tolerated. Slightly higher incidences of adverse events and drug-related adverse events were observed in the efinopegdutide group, primarily related to an imbalance in gastrointestinal adverse events. There were otherwise no meaningful differences between the two treatment groups in the incidence of overall, serious, or drug-related adverse events, including adverse events that led to discontinuation. Dose-dependent gastrointestinal adverse events (predominantly nausea and vomiting) were previously reported in two Phase 2 dose-ranging studies with efinopegdutide in obese patients with and without T2DM^{[0267],[0268]} and have been well described for GLP-1R agonists and other GLP-IR/GCGR co-agonists.^{[0277]} Notably, the two previous Phase 2 dose-ranging studies with efinopegdutide did not employ a titration regimen.^{[0267],[0268]} Because the use of titration regimens to mitigate the occurrence of gastrointestinal adverse events has been successfully implemented for marketed GLP-1 agonists, a titration strategy was used in the current study. The incidences of nausea and vomiting reported in the efinopegdutide group in the present study (27.8% and 16.7%, respectively) were similar to those reported in the semaglutide group (31.5% and 15.1%, respectively) and were notably lower than those reported with efinopegdutide 10 mg in the previous Phase 2 dose-ranging studies (42.9-66.9% and 34.7-55.1%, respectively),^{[0267],[0268]} indicating that the titration strategy was effective in mitigating these adverse events. Effects of efinopegdutide on heart rate and blood pressure in the current study were slightly greater than those observed with semaglutide and were consistent with observations in prior studies with efinopegdutide; these hemodynamic changes did not appear to be mitigated by use of a titration strategy and likely reflect the combined effect of GLP-1 and glucagon agonism.

Similar to what was observed with efinopegdutide in prior Phase 2 studies,^{[0267],[0268]} in the present study there was no meaningful mean change from baseline in A1C or fasting plasma glucose in the efinopegdutide group at Week 24. This was likely due a balanced impact of GLP-1 and glucagon receptor co-agonism on glucose metabolism, resulting in a neutral glycemic effect. The reductions in serum lipids observed with efinopegdutide were also similar to those reported in the prior Phase 2 studies.^{[0267],[0268]} Reductions in hemoglobin have not been reported with GLP-1 receptor agonists and are likely a pharmacologic effect of glucagon agonism. Glucagon has been shown to inhibit erythropoiesis^{Error! Reference source not found.} and to decrease heme production in rodent models,^{[0279]} and normochromic normocytic anemia is common (about 35%) in patients with the glucagonoma syndrome.^{[0280]}

In summary, the study demonstrates that present invention provides a treatment with efinopegdutide that may lead to a significant improvement in LFC, particularly when compared with semaglutide in patients with NAFLD. Treatment with efinopegdutide was also generally well tolerated, with a tolerability profile similar to that of semaglutide. The substantial benefit of therapy with efinopegdutide on reducing liver fat may be due to the complementary dual mechanisms of action of GLP-1 and glucagon receptor agonism, which together lead to improvements in the core pathophysiologic defects associated with NAFLD.

### Compositions of Efinopegdutide

Efinopegdutide may be provided in a composition comprising 93 nmol/mL to 565 nmol/mL of efinopegdutide, 5 mM to 25 mM of a buffering agent selected from citric acid and a salt thereof, acetic acid and a salt thereof, histidine and a salt thereof, phosphoric acid and a salt thereof, and a combination thereof so that the pH of the liquid formulation is 4.8 to 5.5:4% (w/v) to 10% (w/v) of a sugar alcohol; 0.001% (w/v) to 0.1% (w/v) of a nonionic surfactant selected from poloxamer, polysorbate, or a combination thereof; and 0.01-1 mg/mL of L-methionine. In a further embodiment, the composition comprises 274 nmol/mL to 474 nmol/mL of efinopegdutide.

In a further embodiment, the composition comprises 93 nmol/mL to 565 nmol/mL of efinopegdutide in 5-50 mM histidine; 2-15% (w/v) of mannitol; 0.01-1 mg/mL of methionine; and 0.001-0.1% (w/v) of polysorbate 20.

In a further embodiment, the composition comprises 374 nmol/mL of efinopegdutide. 20 mM citric acid-sodium citrate (pH 5.4), 6% mannitol, 0.02% polysorbate 20, and 0.1 mg/mL L-methionine.

In another embodiment, the efinopegdutide may be provided in a composition comprising 93 nmol/mL to 565 nmol/mL of efinopegdutide, 5 mM to 25 mM of a buffering agent selected from citric acid and a salt thereof, acetic acid and a salt thereof, histidine and a salt thereof, phosphoric acid and a salt thereof, and a combination thereof so that the pH of the liquid formulation is 4.8 to 5.5; 4% (w/v) to 10% (w/v) of a saccharide; and 0.01% (w/v) to 0.1% (w/v) of a nonionic surfactant selected from poloxamer, polysorbate, or a combination thereof. In a further embodiment, the composition comprises 274 nmol/mL to 474 nmol/mL of efinopegdutide.

In a further embodiment, the composition comprises 374 nmol/mL of efinopegdutide, 10 mM sodium acetate (pH 5.1), 8.5% sucrose, and 0.02% polysorbate 20.

The efinopegdutide may be provided in a vial as a liquid for use with a syringe or in a vial as a lyophylized powder, which may reconstituted in water for use with a syringe. The efinopegdutide may be provided as a liquid in an injection device designed to provide one or more doses of the composition, each dose comprising up to 10 mg of efinopegdutide. The efinopegdutide may be provided as a liquid in an adjustable injection device designed for the user of the device the ability to select one or more doses of the composition, each dose comprising 2.0, 2.4, 2.5 mg, 5 mg. 7 mg, or 10 mg efinopegdutide.

Specific embodiments of the present invention include:

Embodiment 1. A method for reducing liver fat content (LFC) in an individual with fatty liver disease, comprising:
administering a dose from about 2 mg to about 10 mg of efinopegdutide or a pharmaceutically acceptable salt thereof to the individual once weekly for a time sufficient to reduce the LFC in the individual.

Embodiment 2. The method of Embodiment 1, where the dose is about 2 mg (in alternative embodiments, 2.4 mg) of efinopegdutide or the pharmaceutically acceptable salt thereof.

Embodiment 3. The method of Embodiment 1, where the dose is 2 mg (in alternative embodiments, 2.4 mg) of efinopegdutide or the pharmaceutically acceptable salt thereof.

Embodiment 4. The method of Embodiment 1, where the dose is about 4 mg (in alternative embodiments, 5 mg) of efinopegdutide or the pharmaceutically acceptable salt thereof.

Embodiment 5. The method of Embodiment 1, where the dose is 4 mg (in alternative embodiments, 5 mg) of efinopegdutide or the pharmaceutically acceptable salt thereof.

Embodiment 6. The method of Embodiment 1, where the dose is about 7 mg of efinopegdutide or the pharmaceutically acceptable salt thereof.

Embodiment 7. The method of Embodiment 1, where the dose is 7 mg of efinopegdutide or the pharmaceutically acceptable salt thereof.

Embodiment 8. The method of Embodiment 1, where the dose is about 10 mg of efinopegdutide or the pharmaceutically acceptable salt thereof.

Embodiment 9. The method of Embodiment 1, where the dose is 10 mg of efinopegdutide or the pharmaceutically acceptable salt thereof.

Embodiment 10. The method of any one of Embodiments 1-9, wherein the fatty liver disease is nonalcoholic fatty liver disease (NAFLD) or nonalcoholic steatohepatitis (NASH).

Embodiment 11. The method of Embodiment 10, wherein the NASH is pre-cirrhotic NASH.

Embodiment 12. The method of Embodiment 10, wherein the NASH is cirrhotic NASH.

Embodiment 13. The method of any one of Embodiments 1-12, wherein the time sufficient to reduce the LFC is about 24 weeks.

Embodiment 14. The method of any one of Embodiments 1-13, wherein the individual with fatty liver disease has an LFC of 5% or greater prior to administering the efinopegdutide or pharmaceutically acceptable salt thereof.

Embodiment 15. The method of any one of Embodiments 1-13, wherein the individual with fatty liver disease has an LFC of 10% or greater prior to administering the efinopegdutide or pharmaceutically acceptable salt thereof.

Embodiment 16. The method of any one of Embodiments 1-13, wherein the individual with fatty liver disease has an LFC of 15% or greater prior to administering the efinopegdutide or pharmaceutically acceptable salt thereof.

Embodiment 17. The method of any one of Embodiments 1-13, wherein the individual with fatty liver disease has an LFC of 20% or greater prior to administering the 10 mg of efinopegdutide or pharmaceutically acceptable salt thereof.

Embodiment 18. The method of any one of Embodiments 1-17, wherein the LFC in the individual is reduced to less than 5%.

Embodiment 19 The method of any one of Embodiments 1-18, wherein the individual has type 1 diabetes mellitus (T1DM) or type 2 diabetes mellitus (T2M).

Embodiment 20. The method of Embodiment 19, wherein the individual has type 2 diabetes mellitus (T2M) and is overweight or obese.

Embodiment 21. The method of any one of Embodiments 1-19, wherein the individual is overweight or obese.

Embodiment 22. A method for treating fatty liver disease in an individual in need thereof, comprising:
administering a dose from about 2 mg to about 10 mg of efinopegdutide or a pharmaceutically acceptable salt thereof to the individual once weekly for a time sufficient to treat the fatty liver disease in the individual.

Embodiment 23. The method of Embodiment 22, where the dose is about 2 mg (in alternative embodiments, 2.4 mg) of efinopegdutide or the pharmaceutically acceptable salt thereof.

Embodiment 24. The method of Embodiment 22, where the dose is 2 mg (in alternative embodiments, 2.4 mg) of efinopegdutide or the pharmaceutically acceptable salt thereof.

Embodiment 25. The method of Embodiment 22, where the dose is about 4 mg (in alternative embodiments, 5 mg) of efinopegdutide or the pharmaceutically acceptable salt thereof.

Embodiment 26. The method of Embodiment 22, where the dose is 4 mg (in alternative embodiments, 5 mg) of efinopegdutide or the pharmaceutically acceptable salt thereof.

Embodiment 27. The method of Embodiment 22, where the dose is about 7 mg of efinopegdutide or the pharmaceutically acceptable salt thereof.

Embodiment 28. The method of Embodiment 22, where the dose is 7 mg of efinopegdutide or the pharmaceutically acceptable salt thereof.

Embodiment 29. The method of Embodiment 22, where the dose is about 10 mg of efinopegdutide or the pharmaceutically acceptable salt thereof.

Embodiment 30. The method of Embodiment 22, where the dose is 10 mg of efinopegdutide or the pharmaceutically acceptable salt thereof.

Embodiment 31. The method of any one of Embodiments 22-30, wherein the fatty liver disease is nonalcoholic fatty liver disease (NAFLD) or nonalcoholic steatohepatitis (NASH).

Embodiment 32. The method of Embodiment 31, wherein the NASH is pre-cirrhotic NASH.

Embodiment 33. The method of Embodiment 31, wherein the NASH is cirrhotic NASH.

Embodiment 34. The method of any one of Embodiments 22-33, wherein the time sufficient to treat the fatty liver disease is about 24 weeks.

Embodiment 35. The method of any one of Embodiments 22-34, wherein the individual with fatty liver disease has a liver fat content (LFC) of 5% or greater prior to administering the efinopegdutide or pharmaceutically acceptable salt thereof.

Embodiment 36. The method of any one of Embodiments 22-34, wherein the individual with fatty liver disease has an LFC of 10% or greater prior to administering the efinopegdutide or pharmaceutically acceptable salt thereof.

Embodiment 37. The method of any one of Embodiments 22-34, wherein the individual with fatty liver disease has an LFC of 15% or greater prior to administering the efinopegdutide or pharmaceutically acceptable salt thereof.

Embodiment 38. The method of any one of Embodiments 22-34, wherein the individual with fatty liver disease has an LFC of 20% or greater prior to administering the 10 mg of efinopegdutide or pharmaceutically acceptable salt thereof.

Embodiment 39. The method of any one of Embodiments 22-38, wherein the LFC in the individual is reduced to less than 5%.

Embodiment 40 The method of any one of Embodiments 22-39, wherein the individual has type 1 diabetes mellitus (TTDM) or type 2 diabetes mellitus (T2M).

Embodiment 41. The method of Embodiment 40, wherein the individual has type 2 diabetes mellitus (T2M) and is overweight or obese.

Embodiment 42. The method of any one of Embodiments 22-40, wherein the individual is overweight or obese.

Embodiment 43. A method for reducing liver fat content (LFC) to less than 5% in an individual with elevated LFC comprising in the following order the steps of
(a) administering to the individual a dose of about 2.0 mg (or in alternative embodiments, about 2.4 mg) of efinopegdutide or a pharmaceutically acceptable salt thereof once a week for four weeks,
(b) administering to the individual a dose of about 5.0 mg of efinopegdutide or a pharmaceutically acceptable salt thereof once a week for four weeks, and
(c) administering to the individual a dose of about 10 mg of efinopegdutide or a pharmaceutically acceptable salt thereof once a week for a time sufficient to reduce the LFC to less than 5%.

Embodiment 44. The method of Embodiment 43, comprising in the following order the steps of:
(a) administering to the individual a dose of 2.0 mg (in alternative embodiments, 2.4 mg) of efinopegdutide or a pharmaceutically acceptable salt thereof once a week for four weeks,
(b) administering to the individual a dose of 5.0 mg of efinopegdutide or a pharmaceutically acceptable salt thereof once a week for four weeks, and
(c) administering to the individual a dose of 10 mg of efinopegdutide or a pharmaceutically acceptable salt thereof once a week for a time sufficient to reduce the LFC to less than 5%.

Embodiment 45. A method for reducing liver fat content (LFC) to less than 5% in an individual with elevated LFC comprising in the following order the steps of
(a) administering to the individual a dose of about 2.0 mg of efinopegdutide (in alternative embodiments, 2.4 mg) or a pharmaceutically acceptable salt thereof once a week for four weeks, and
(b) administering to the individual a dose of about 4.0 mg of efinopegdutide or a pharmaceutically acceptable salt thereof once a week for 48 weeks.

Embodiment 46. The method of Embodiment 45, comprising in the following order the steps of
(a) administering to the individual a dose of 2.0 mg of efinopegdutide (in alternative embodiments, 2.4 mg) or a pharmaceutically acceptable salt thereof once a week for four weeks, and
(b) administering to the individual a dose of 4.0 mg of efinopegdutide or a pharmaceutically acceptable salt thereof once a week for 48 weeks.

Embodiment 47. A method for reducing liver fat content (LFC) to less than 5% in an individual with elevated LFC comprising in the following order the steps of:
(a) administering to the individual a dose of about 2.0 mg of efinopegdutide (in alternative embodiments, 2.4 mg) or a pharmaceutically acceptable salt thereof once a week for four weeks,
(b) administering to the individual a dose of about 4.0 mg of efinopegdutide or a pharmaceutically acceptable salt thereof once a week for four weeks, and
(c) administering to the individual a dose of about 7 mg of efinopegdutide or a pharmaceutically acceptable salt thereof once a week for 44 weeks.

Embodiment 48. The method of Embodiment 47, comprising in the following order the steps of:
(a) administering to the individual a dose of 2.0 mg of efinopegdutide (in alternative embodiments, 2.4 mg) or a pharmaceutically acceptable salt thereof once a week for four weeks,
(b) administering to the individual a dose of 4.0 mg of efinopegdutide or a pharmaceutically acceptable salt thereof once a week for four weeks, and
(c) administering to the individual a dose of 7 mg of efinopegdutide or a pharmaceutically acceptable salt thereof once a week for 44 weeks.

Embodiment 49. A method for reducing liver fat content (LFC) to less than 5% in an individual with elevated LFC comprising in the following order the steps of:
(a) administering to the individual a dose of about 2.0 mg of efinopegdutide (in alternative embodiments, 2.4 mg) or a pharmaceutically acceptable salt thereof once a week for four weeks,
(b) administering to the individual a dose of about 4.0 mg of efinopegdutide or a pharmaceutically acceptable salt thereof once a week for four weeks,
(c) administering to the individual a dose of about 7.0 mg of efinopegdutide or a pharmaceutically acceptable salt thereof once a week for four weeks, and
(d) administering to the individual a dose of about 10.0 mg of efinopegdutide or a pharmaceutically acceptable salt thereof once a week for 40 weeks.

Embodiment 50. The method of Embodiment 49, comprising in the following order the steps of:
(a) administering to the individual a dose of 2.0 mg of efinopegdutide (in alternative embodiments, 2.4 mg) or a pharmaceutically acceptable salt thereof once a week for four weeks.
(b) administering to the individual a dose of 4.0 mg of efinopegdutide or a pharmaceutically acceptable salt thereof once a week for four weeks,
(c) administering to the individual a dose of 7.0 mg of efinopegdutide or a pharmaceutically acceptable salt thereof once a week for four weeks, and
(d) administering to the individual a dose of 10.0 mg of efinopegdutide or a pharmaceutically acceptable salt thereof once a week for 40 weeks.

Embodiment 51. The method of any of Embodiments 43-50, wherein the individual with elevated LFC has fatty liver disease.

Embodiment 52. The method of Embodiment 51, wherein the fatty liver disease is nonalcoholic fatty liver disease (NAFLD) or nonalcoholic steatohepatitis (NASH).

Embodiment 53. The method of Embodiment 52, wherein the NASH is pre-cirrhotic NASH.

Embodiment 54. The method of Embodiment 52, wherein the NASH is cirrhotic NASH.

Embodiment 55. The method of Embodiment 43 or Embodiment 44, wherein the time sufficient to reduce the liver fat to less than 5% is about 24 weeks.

Embodiment 56. The method of any one of Embodiments 43-55, wherein the individual with fatty liver disease has an LFC of 5% or greater prior to beginning the method.

Embodiment 57. The method of any one of Embodiments 43-55, wherein the individual with fatty liver disease has an LFC of 10% or greater prior to beginning the method.

Embodiment 58. The method of any one of Embodiments 43-55, wherein the individual with fatty liver disease has an LFC of 15% or greater prior to beginning the method.

Embodiment 59. The method of any one of Embodiments 43-55, wherein the individual with fatty liver disease has an LFC of 20% or greater prior to beginning the method.

Embodiment 60. The method of any one of Embodiments 43-59, wherein the individual has type 1 diabetes mellitus (T1DM) or type 2 diabetes mellitus (T2M).

Embodiment 61. The method of Embodiment 60, wherein the individual has type 2 diabetes mellitus (T2M) and is overweight or obese.

Embodiment 62. The method of any one of Embodiments 43-60, wherein the individual is overweight or obese.

Embodiment 63. A method for treating fatty liver disease in an individual in need thereof comprising in the following order the steps of
(a) administering to the individual a dose of about 2.0 mg of efinopegdutide (in alternative embodiments, 2.4 mg) or a pharmaceutically acceptable salt thereof once a week for four weeks.
(b) administering to the individual a dose of about 5.0 mg of efinopegdutide or a pharmaceutically acceptable salt thereof once a week for four weeks, and
(c) administering to the individual a dose of about 10 mg of efinopegdutide or a pharmaceutically acceptable salt thereof once a week for a time sufficient to treat the fatty liver disease in the individual.

Embodiment 64. The method of Embodiment 63, comprising in the following order the steps of:
(a) administering to the individual a dose of 2.0 mg of efinopegdutide (in alternative embodiments, 2.4 mg) or a pharmaceutically acceptable salt thereof once a week for four weeks,
(b) administering to the individual a dose of 5.0 mg of efinopegdutide or a pharmaceutically acceptable salt thereof once a week for four weeks, and
(c) administering to the individual a dose of 10 mg of efinopegdutide or a pharmaceutically acceptable salt thereof once a week for a time sufficient to reduce the LFC to less than 5%.

Embodiment 65. A method for treating fatty liver disease in an individual in need thereof comprising in the following order the steps of
(a) administering to the individual a dose of about 2.0 mg of efinopegdutide (in alternative embodiments, 2.4 mg) or a pharmaceutically acceptable salt thereof once a week for four weeks, and
(b) administering to the individual a dose of about 4.0 mg of efinopegdutide or a pharmaceutically acceptable salt thereof once a week for 48 weeks.

Embodiment 66. The method of Embodiment 65, comprising in the following order the steps of
(a) administering to the individual a dose of 2.0 mg of efinopegdutide (in alternative embodiments, 2.4 mg) or a pharmaceutically acceptable salt thereof once a week for four weeks, and
(b) administering to the individual a dose of 4.0 mg of efinopegdutide or a pharmaceutically acceptable salt thereof once a week for 48 weeks.

Embodiment 67. A method for treating fatty liver disease in an individual in need thereof comprising in the following order the steps of
(a) administering to the individual a dose of about 2.0 mg of efinopegdutide (in alternative embodiments, 2.4 mg) or a pharmaceutically acceptable salt thereof once a week for four weeks,
(b) administering to the individual a dose of about 4.0 mg of efinopegdutide or a pharmaceutically acceptable salt thereof once a week for four weeks, and
(c) administering to the individual a dose of about 7 mg of efinopegdutide or a pharmaceutically acceptable salt thereof once a week for 44 weeks.

Embodiment 68. The method of Embodiment 67, comprising in the following order the steps of
(a) administering to the individual a dose of 2.0 mg of efinopegdutide (in alternative embodiments, 2.4 mg) or a pharmaceutically acceptable salt thereof once a week for four weeks,
(b) administering to the individual a dose of 4.0 mg of efinopegdutide or a pharmaceutically acceptable salt thereof once a week for four weeks, and
(c) administering to the individual a dose of 7 mg of efinopegdutide or a pharmaceutically acceptable salt thereof once a week for 44 weeks.

Embodiment 69. A method for treating fatty liver disease in an individual in need thereof comprising in the following order the steps of:
(a) administering to the individual a dose of about 2.0 mg of efinopegdutide (in alternative embodiments, 2.4 mg) or a pharmaceutically acceptable salt thereof once a week for four weeks,
(b) administering to the individual a dose of about 4.0 mg of efinopegdutide or a pharmaceutically acceptable salt thereof once a week for four weeks,
(c) administering to the individual a dose of about 7.0 mg of efinopegdutide or a pharmaceutically acceptable salt thereof once a week for four weeks, and
(d) administering to the individual a dose of about 10.0 mg of efinopegdutide or a pharmaceutically acceptable salt thereof once a week for 40 weeks.

Embodiment 70. The method of Embodiment 69, comprising in the following order the steps of
(a) administering to the individual a dose of 2.0 mg of efinopegdutide (in alternative embodiments, 2.4 mg) or a pharmaceutically acceptable salt thereof once a week for four weeks,
(b) administering to the individual a dose of 4.0 mg of efinopegdutide or a pharmaceutically acceptable salt thereof once a week for four weeks,
(c) administering to the individual a dose of 7.0 mg of efinopegdutide or a pharmaceutically acceptable salt thereof once a week for four weeks, and
(d) administering to the individual a dose of 10.0 mg of efinopegdutide or a pharmaceutically acceptable salt thereof once a week for 40 weeks.

Embodiment 71. The method of any of Embodiments 63-70, wherein the individual with fatty liver disease has elevated liver fat content (LFC).

Embodiment 72. The method of Embodiment 71, wherein the fatty liver disease is nonalcoholic fatty liver disease (NAFLD) or nonalcoholic steatohepatitis (NASH).

Embodiment 73. The method of Embodiment 72, wherein the NASH is pre-cirrhotic NASH.

Embodiment 74. The method of Embodiment 72, wherein the NASH is cirrhotic NASH.

Embodiment 75. The method of Embodiment 63 or Embodiment 64, wherein the time sufficient to treat the fatty liver disease in the individual is about 24 weeks.

Embodiment 76. The method of any one of Embodiments 63-75, wherein the individual with fatty liver disease has an LFC of 5% or greater prior to beginning the method.

Embodiment 77. The method of any one of Embodiments 63-75, wherein the individual with fatty liver disease has an LFC of 10% or greater prior to beginning the method.

Embodiment 78. The method of any one of Embodiments 63-75, wherein the individual with fatty liver disease has an LFC of 15% or greater prior to beginning the method.

Embodiment 79. The method of any one of Embodiments 63-75, wherein the individual with fatty liver disease has an LFC of 20% or greater prior to beginning the method.

Embodiment 80. The method of any one of Embodiments 63-79, wherein the individual has type 1 diabetes mellitus (T1DM) or type 2 diabetes mellitus (T2M).

Embodiment 81. The method of Embodiment 80, wherein the individual has type 2 diabetes mellitus (T2M) and is overweight or obese.

Embodiment 82. The method of any one of Embodiments 63-80, wherein the individual is overweight or obese.

Embodiment 83. Use of efinopegdutide or a pharmaceutically acceptable salt thereof for the manufacture of a medicament for reducing liver fat content (LFC) in individuals with elevated LFC to a LFC of less than 5%.

Embodiment 84. The use of Embodiment 83, wherein the medicament comprises about 2 mg of efinopegdutide or the pharmaceutically acceptable salt thereof.

Embodiment 85. The use of Embodiment 83, wherein the medicament comprises 2 mg of efinopegdutide or the pharmaceutically acceptable salt thereof.

Embodiment 86. The use of Embodiment 83, wherein the medicament comprises about 4 mg of efinopegdutide or the pharmaceutically acceptable salt thereof.

Embodiment 87. The use of Embodiment 83, wherein the medicament comprises 4 mg of efinopegdutide or the pharmaceutically acceptable salt thereof.

Embodiment 88. The use of Embodiment 83, wherein the medicament comprises about 7 mg of efinopegdutide or the pharmaceutically acceptable salt thereof.

Embodiment 89. The use of Embodiment 83, wherein the medicament comprises 7 mg of efinopegdutide or the pharmaceutically acceptable salt thereof.

Embodiment 90. The use of Embodiment 83, wherein the medicament comprises about 10 mg of efinopegdutide or the pharmaceutically acceptable salt thereof.

Embodiment 91. The use of Embodiment 83, wherein the medicament comprises 10 mg of efinopegdutide or the pharmaceutically acceptable salt thereof.

Embodiment 92. Use of efinopegdutide or a pharmaceutically acceptable salt thereof for the manufacture of a medicament for the treatment of fatty liver disease in individuals in need thereof.

Embodiment 93. The use of Embodiment 92, wherein the fatty liver disease is nonalcoholic fatty liver disease (NAFLD) or nonalcoholic steatohepatitis (NASH).

Embodiment 94. The use of Embodiment 93, wherein the NASH is pre-cirrhotic NASH.

Embodiment 95. The use of Embodiment 93, wherein the NASH is cirrhotic NASH.

Embodiment 96. The use of Embodiment 93, wherein the individual with fatty liver disease has a liver fat content (LFC) of 5% or greater prior to administering the efinopegdutide or pharmaceutically acceptable salt thereof.

Embodiment 97. The use of any one of Embodiments 92-96, wherein the medicament comprises about 2 mg of efinopegdutide or the pharmaceutically acceptable salt thereof.

Embodiment 98. The use of any one of Embodiments 92-96, wherein the medicament comprises 2 mg of efinopegdutide or the pharmaceutically acceptable salt thereof.

Embodiment 99. The use of any one of Embodiments 92-96, wherein the medicament comprises about 4 mg of efinopegdutide or the pharmaceutically acceptable salt thereof.

Embodiment 100. The use of any one of Embodiments 92-96, wherein the medicament comprises 4 mg of efinopegdutide or the pharmaceutically acceptable salt thereof.

Embodiment 101. The use of any one of Embodiments 92-96, wherein the medicament comprises about 7 mg of efinopegdutide or the pharmaceutically acceptable salt thereof.

Embodiment 102. The use of any one of Embodiments 92-96, wherein the medicament comprises 7 mg of efinopegdutide or the pharmaceutically acceptable salt thereof.

Embodiment 103. The use of any one of Embodiments 92-96, wherein the medicament comprises about 10 mg of efinopegdutide or the pharmaceutically acceptable salt thereof.

Embodiment 104. The use of any one of Embodiments 92-96, wherein the medicament comprises 10 mg of efinopegdutide or the pharmaceutically acceptable salt thereof.

Embodiment 105. Efinopegdutide or a pharmaceutically acceptable salt thereof for reducing liver fat content (LFC) in individuals with elevated LFC to a LFC of less than 5%.

Embodiment 106. The efinopegdutide or pharmaceutically acceptable salt thereof of Embodiment 105, wherein the efinopegdutide or pharmaceutically acceptable salt thereof is provided in about 2 mg doses (in alternative embodiments, 2.4 mg).

Embodiment 107. The efinopegdutide or pharmaceutically acceptable salt thereof of Embodiment 105, wherein the efinopegdutide or pharmaceutically acceptable salt thereof is provided in 2 mg doses (in alternative embodiments, 2.4 mg).

Embodiment 108. The efinopegdutide or pharmaceutically acceptable salt thereof of Embodiment 105, wherein the efinopegdutide or pharmaceutically acceptable salt thereof is provided in about 4 mg doses.

Embodiment 109. The efinopegdutide or pharmaceutically acceptable salt thereof of Embodiment 105, wherein the efinopegdutide or pharmaceutically acceptable salt thereof is provided in 4 mg doses.

Embodiment 110. The efinopegdutide or pharmaceutically acceptable salt thereof of Embodiment 105, wherein the efinopegdutide or pharmaceutically acceptable salt thereof is provided in about 7 mg doses.

Embodiment 111. The efinopegdutide or pharmaceutically acceptable salt thereof of Embodiment 105, wherein the efinopegdutide or pharmaceutically acceptable salt thereof is provided in 7 mg doses.

Embodiment 112. The efinopegdutide or pharmaceutically acceptable salt thereof of Embodiment 105, wherein the efinopegdutide or pharmaceutically acceptable salt thereof is provided in about 10 mg doses.

Embodiment 113. The efinopegdutide or pharmaceutically acceptable salt thereof of Embodiment 105, wherein the efinopegdutide or pharmaceutically acceptable salt thereof is provided in 10 mg doses.

Embodiment 114. A pharmaceutical composition comprising about 2 mg efinopegdutide (in alternative embodiments. 2.4 mg) or a pharmaceutically acceptable salt thereof and a pharmaceutically acceptable carrier.

Embodiment 115. A pharmaceutical composition comprising 2 mg efinopegdutide (in alternative embodiments, 2.4 mg) or a pharmaceutically acceptable salt thereof and a pharmaceutically acceptable carrier.

Embodiment 116. A pharmaceutical composition comprising about 4 mg efinopegdutide or a pharmaceutically acceptable salt thereof and a pharmaceutically acceptable carrier.

Embodiment 117. A pharmaceutical composition comprising 4 mg efinopegdutide or a pharmaceutically acceptable salt thereof and a pharmaceutically acceptable carrier.

Embodiment 118. A pharmaceutical composition comprising about 7 mg efinopegdutide or a pharmaceutically acceptable salt thereof and a pharmaceutically acceptable carrier.

Embodiment 119. A pharmaceutical composition comprising 7 mg efinopegdutide or a pharmaceutically acceptable salt thereof and a pharmaceutically acceptable carrier.

Embodiment 120. A pharmaceutical composition comprising about 10 mg efinopegdutide or a pharmaceutically acceptable salt thereof and a pharmaceutically acceptable carrier.

Embodiment 121. A pharmaceutical composition comprising 10 mg efinopegdutide or a pharmaceutically acceptable salt thereof and a pharmaceutically acceptable carrier.

Embodiment 121A. The pharmaceutical composition of any of Embodiments 112-121 comprising from about 93 nmol/mL to about 565 nmol/mL (in specific embodiments, from 93 nmol/mL to 565 nmol/mL) of efinopegdutide; from about 5 mM to about 25 mM (in specific embodiments, from 5 mM to 25 mM) of a buffering agent selected from the group consisting of citric acid or a pharmaceutically acceptable salt thereof, acetic acid or a pharmaceutically acceptable salt thereof, histidine or a pharmaceutically acceptable salt thereof, phosphoric acid or a pharmaceutically acceptable a salt thereof, and a combination of the foregoing.

Embodiment 121B. The pharmaceutical composition of Embodiment 121A wherein the pH of the liquid formulation is from about 4.8 to about 5.5 (in specific embodiments, from 4.8 to 5.5); from about 4% (w/v) to about 10% (w/v) (in specific embodiments from 4% (w/v) to 10% (w/v)) of a sugar alcohol; from about 0.001% (w/v) to about 0.1% (w/v) (in specific embodiments, from 0.001% (w/v) to 0.1% (w/v)) of a nonionic surfactant selected from the group consisting of: a poloxamer, polysorbate, or a combination of the foregoing; and from about 0.01 to about 1 mg/mL (in specific embodiments, from 0.01 to 1 mg/mL) of L-methionine.

Embodiment 121C. The pharmaceutical composition of Embodiment 121A or Embodiment 121B comprising from about 274 nmol/mL to about 474 nmol/mL (in specific embodiments, from 274 nmol/mL to 474 nmol/mL) of efinopegdutide or the pharmaceutically acceptable salt thereof.

Embodiment 121D. The pharmaceutical composition of Embodiment 121 comprising from about 93 nmol/mL to about 565 nmol/mL (in specific embodiments, from 93 nmol/mL to 565 nmol/mL) of efinopegdutide or the pharmaceutically acceptable salt thereof, from about 5-50 mM (in specific embodiments from 5-50 mM) histidine; from about 2 to about 15% (w/v) (in specific embodiments from 2 to 15% (w/v)) of mannitol; from about 0.01 to about 1 mg/mL (in specific embodiments, from 0.01 to 1 mg/mL) of methionine; and from about 0.001 to about 0.1% (w/v) (in specific embodiments, from 0.001 to 0.1% (w/v)) of polysorbate 20.

Embodiment 121E. The pharmaceutical composition of Embodiment 121 comprising about 374 nmol/mL (in specific embodiments, 374 nmol/mL) of efinopegdutide or the pharmaceutically acceptable salt thereof; about 20 mM (in specific embodiments, 20 mM) citric acid-sodium citrate; about 6% (w/v) (in specific embodiments, 6% (w/v)) of mannitol: about 0.1 mg/mL (in specific embodiments, 0.1 mg/mL) of methionine; and about 0.02% (w/v) (in specific embodiments, 0.02% (w/v)) of polysorbate 20, wherein the pH is about 5.4 (in specific embodiments, 5.4).

Embodiment 121F. The pharmaceutical composition of Embodiment 121 comprising from about 93 nmol/mL to about 565 nmol/mL (in specific embodiments, from 93 nmol/mL to 565 nmol/mL) of efinopegdutide or the pharmaceutically acceptable salt thereof; from about 5 mM to about 25 mM (in specific embodiments, from 5 mM to 25 mM) of a buffering agent selected from the group consisting of: citric acid or a pharmaceutically acceptable salt thereof, acetic acid or a pharmaceutically acceptable salt thereof, histidine or a pharmaceutically acceptable salt thereof, phosphoric acid or a pharmaceutically acceptable salt thereof, or a combination of the foregoing; from about 4% (w/v) to about 10% (w/v) (in specific embodiments, from 4% (w/v) to 10% (w/v)) of a saccharide; and from about 0.01% (w/v) to about 0.1% (w/v) (in specific embodiments, from 0.01% (w/v) to 0.1% (w/v)) of a nonionic surfactant selected from the group consisting of: a poloxamer, polysorbate, or a combination of the foregoing; wherein the pH is from about 4.8 to about 5.5 (in specific embodiment, from 4.8 to 5.5).

Embodiment 121G. The pharmaceutical composition of Embodiment 121G comprising from about 274 nmol/mL to about 474 nmol/mL (in specific embodiments, from 274 to 474 nmol/mL) of efinopegdutide or the pharmaceutically acceptable salt thereof.

Embodiment 121H. The pharmaceutical composition of Embodiment 121 comprising about 374 nmol/mL (in specific embodiments, 374 nmol/mL) of efinopegdutide or the pharmaceutically acceptable salt thereof, about 10 mM (in specific embodiments, 10 mM) sodium acetate, about 8.5% (in specific embodiments, 8.5%) sucrose, and about 0.02% (w/v) (in specific embodiments, 0.02% (w/v) polysorbate 20), wherein the pH is about 5.1 (in specific embodiments, 5.1).

Embodiments 121-121H are distinct embodiments of Embodiment 121 and referred to separately in the following Embodiments.

Embodiment 122. The method of any of Embodiments 1-82 wherein the efinopegdutide or pharmaceutically acceptable salt thereof is administered subcutaneously.

Embodiment 123. The use of any of Embodiments 83-104 wherein the medicament is for subcutaneous administration.

Embodiment 124. The efinopegdutide or pharmaceutically acceptable salt thereof of any of Embodiments 105-113 for subcutaneous administration.

Embodiment 125. The pharmaceutical composition of any of Embodiments 114-121 for subcutaneous administration.

Embodiment 126. The method, use, efinopegdutide or pharmaceutically acceptable salt thereof, or pharmaceutical composition of any of Embodiments 1-125 wherein there is a reduction from baseline in LFC in the individual (or an individual) at Week 24 of treatment of about ≥30%, about ≥50%, or about ≥70% (or in particular embodiments ≥30%, ≥50% or ≥70%).

Embodiment 127. The method, use, efinopegdutide or pharmaceutically acceptable salt thereof, or pharmaceutical composition of any of Embodiments 1-125 wherein there is a reduction from body weight from baseline in the individual (or an individual) at Week 24 of treatment of ≥about 5%, >about 5% to ≤about 10%, or >about 10% (or in particular embodiments ≥5%, >5% to ≤10%, or >10%).

The following examples are intended to promote a further understanding of the present invention.

### Example 1

The Phase 2a study in this Example was conducted to assess the efficacy and safety of GLP-1 and glucagon receptor co-agonism with efinopegdutide relative to GLP-1 agonism alone with semaglutide in patients with NAFLD with and without T2DM, and to inform on the potential of efinopegdutide as a novel therapy for NASH. The results herein demonstrate that treatment of NAFLD patients with efinopegdutide markedly reduced their liver fat content compared to semaglutide, suggesting that efinopegdutide may be an effective treatment for NASH.

### Participant Selection

This study enrolled males and females aged 18 to 70 years. Inclusion criteria included NAFLD based on an LFC of 10% or greater as assessed by magnetic resonance imaging-estimated proton density fat fraction (MRI-PDFF), body mass index (BMI) 25 kg/m² or greater and 50 kg/m² or less, stable body weight (based on self-reporting) defined as 5% or less gain or loss of body weight for at least three months before screening, and either no history of T2DM or a history of T2DM with an AlC 8.5% or less at screening and controlled by diet and/or a stable dose of metformin for the three months before screening. Antihyperglycemic agents other than metformin were not permitted.

Key exclusion criteria included history or evidence of chronic liver disease other than NAFLD or NASH: known history of cirrhosis (fibrosis stage greater than three based on a historical liver biopsy or a liver stiffness score greater than 14 kPa based on a historical FibroScan® assessment (FibroScan® is a noninvasive diagnostic ultrasound-based device used to measure liver scarring, or fibrosis, caused by different liver diseases): decompensated liver disease including, but not limited to, history of ascites, esophageal or gastric variceal bleeding, hepatocellular carcinoma, hepatic encephalopathy, splenomegaly, or spontaneous bacterial peritonitis; treatment with any GLP-1 receptor agonist or investigational GLP-1/glucagon receptor co-agonist within six months before screening; treatment with thiazolidinediones (i.e., pioglitazone, rosiglitazone) within six months before screening: previous or current use of prescription weight-management medications or over-the-counter weight-loss medications or therapies within the three months before screening; treatment with an antihyperlipidemic therapy that was not at a stable dose for at least one month before screening; treatment with greater than 100 international units (IU)/day of vitamin E at a dose that was not stable for at least three months before screening.

### Study Design

This was a Phase 2a, randomized, active-comparator-controlled (semaglutide; marketed under the trade name Ozempic*, Novo Nordisk, DK), parallel-group, multi-site, open-label study of efinopegdutide in participants with NAFLD (protocol 001; EudraCT: 2020-005136-30; NCT: 04944992: ClinicalTrials.gov. Jun. 30, 2021). The study protocol was approved by the institutional review board or independent ethics committee at each investigational site and was conducted in accordance with applicable regulations and the ethical principles of Good Clinical Practice as defined by the International Conference on Harmonization and Declaration of Helsinki. Written informed consent was obtained from all participants.

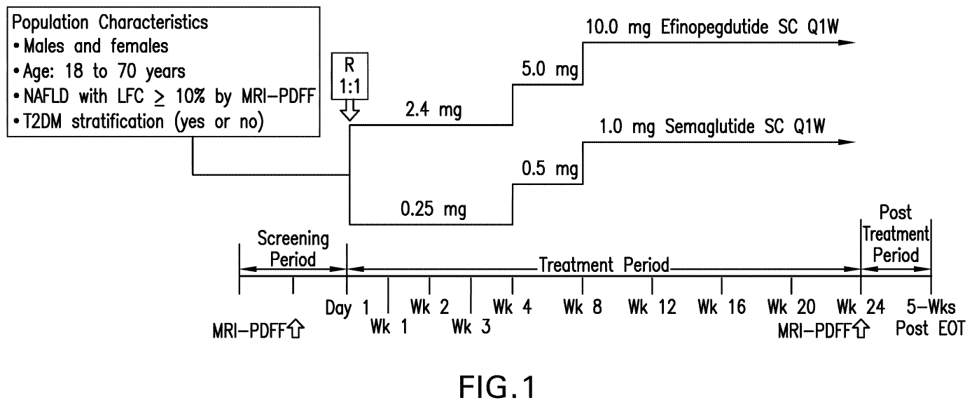

The study design is shown in FIG. 1. The duration of the study was approximately 32 weeks, which included a staged screening period of approximately four weeks, a 24-week active-comparator-controlled treatment period, and a post-treatment period follow-up visit at approximately five weeks after the last dose of study intervention.

During the screening period, Magnetic Resonance Imaging-Proton Density Fat Fraction (MRI-PDFF) was performed on all participants who met other screening eligibility requirements. If a participant had an LFC as assessed by MRI-PDFF of 10% or greater as determined by blinded independent central review (BICR) and all other eligibility criteria had been met, the participant proceeded to randomization.

At baseline (Day 1), participants who met eligibility criteria were randomly assigned in a 1:1 ratio to open-label efinopegdutide 10 mg SC once weekly or semaglutide 1 mg SC once weekly, stratified according to concurrent diagnosis of T2DM at the time of randomization.

Participants randomized to efinopegdutide 10 mg once weekly or semaglutide 1 mg once weekly followed a three-step dose-escalation regimen to achieve the planned study dose. Participants randomized to the efinopegdutide 10 mg once weekly group were started at a dose of 2.4 mg once weekly from Day 1 to Week 4: the dose was increased to 5 mg QIW from Week 4 up to Week 8 and then to 10 mg once weekly from Week 8 to Week 24. Participants who could not tolerate the efinopegdutide 10 mg once weekly dose could continue in the study on the efinopegdutide 5 mg once weekly dose. Down-titration below the 5 mg once weekly dose for efinopegdutide was not permitted; participants unable to tolerate at least the 5 mg once weekly efinopegdutide dose were discontinued from study treatment. Participants randomized to the semaglutide 1 mg once weekly treatment group were started at a dose of 0.25 mg once weekly from Day 1 to Week 4: the dose was increased to 0.5 mg once weekly from Week 4 to Week 8 and then to 1 mg once weekly from Week 8 to Week 24. Participants who could not tolerate the semaglutide 1 mg once weekly dose could continue in the study on the semaglutide 0.5 mg once weekly dose. Down-titration below the 0.5 mg once weekly dose for semaglutide was not permitted; participants unable to tolerate at least the 0.5 mg once weekly semaglutide dose were discontinued from study treatment.

### Efficacy Endpoints

The primary efficacy endpoint was the relative reduction from baseline in LFC measured by MRI-PDFF (evaluated by BICR) after 24 weeks of treatment. Secondary efficacy endpoints included the percent change from baseline in body weight and fasting lipid levels (total cholesterol, high-density lipoprotein cholesterol, low-density lipoprotein cholesterol, and triglycerides) after 24 weeks of treatment. The following exploratory endpoints supported the primary efficacy endpoint: the proportions of participants with relative reductions from baseline in LFC of 30% or greater, ≥0% or greater and 70% or greater; the proportion of participants who had achieved normal LFC (less than 5%); and the mean relative reduction from baseline in LFC by weight-loss categories (percent reductions from baseline in body weight of ≤% or less, greater than 5% to 10% or less, and greater than 10%).

### Safety Assessments

The safety and tolerability of efinopegdutide were monitored throughout the study by clinical evaluation of adverse events and inspection of other study parameters including physical examinations, 12-lead electrocardiograms (ECGs), vital signs, and laboratory safety tests.

### Statistical Analysis

The efficacy analysis population included all randomized participants who received at least one dose of study intervention and had at least one assessment.

The primary efficacy analysis compared the efficacy of efinopegdutide to semaglutide in relative reduction from baseline in LFC at Week 24. The difference (efinopegdutide minus semaglutide) in means and the associated 90% confidence interval (CI) and p-value were provided based on a longitudinal data analysis (LDA) model.^{[0269]} Efinopegdutide was considered superior to semaglutide if the 1-sided p-value was <0.05. The model-based least squares (LS) mean change from baseline for each treatment group and difference (with CI) between-treatment groups at the Week 24 post-baseline time point were summarized.

Percent changes in body weight and fasting lipid levels were analyzed using the same LDA model as described for the primary endpoint.

The safety analysis population consisted of all randomized participants who received at least one dose of study intervention.

A sample size of 65 participants per arm provided approximately 99% power to establish that efinopegdutide was superior to semaglutide, with a 1-sided α=0.05, assuming a true treatment difference of approximately 19.4%, a common standard deviation (SD) of 20%, and 10% discontinuation.

### Results

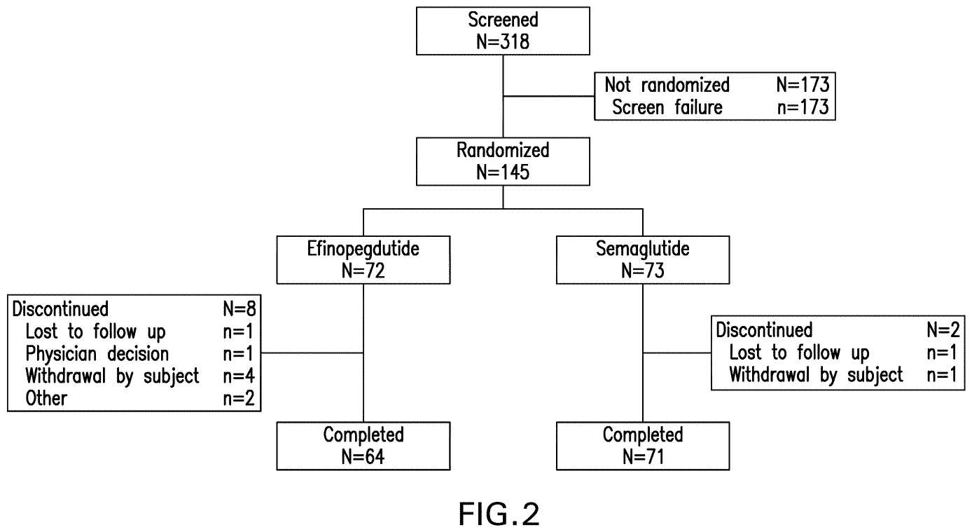

This study was performed from 4 Aug. 2021 to 19 Oct. 2022 across 79 centers in 16 countries (Argentina, Australia, Canada, France, Israel, Italy, Korea, Mexico, New Zealand, Poland, Russia, Spain, Taiwan, Turkey, Ukraine, and United States). The disposition of participants is shown in FIG. 2. A total of 145 participants were randomized to treatment, of which 135 completed the study.

### Demographics and Baseline Characteristics

The two treatment groups had similar demographics and baseline characteristics (Table 1).

TABLE 1
Demographics and baseline characteristics

| | Efinopegdutide | Semaglutide | Total |
|---|---|---|---|
| Parameter | N = 72 | N = 73 | N = 145 |
| Sex, n (%) | | | |
| Male | 39 (54.2) | 41 (56.2) | 80 (55.2) |
| Female | 33 (45.8) | 32 (43.8) | 65 (44.8) |
| Mean (SD) Age, years | 48.1 (11.0) | 50.9 (10.9) | 49.5 (11.0) |
| Race, n (%) | | | |
| American Indian or | 3 (4.2) | 2 (2.7) | 5 (3.4) |
| Alaska Native | | | |
| Asian | 7 (9.7) | 7 (9.6) | 14 (9.7) |
| Black | 0 | 1 (1.4) | 1 (0.7) |
| White | 62 (86.1) | 63 (86.3) | 125 (86.2) |
| Ethnicity, n (%) | | | |
| Hispanic Or Latino | 25 (34.7) | 26 (35.6) | 51 (35.2) |
| Not Hispanic Or | 46 (63.9) | 47 (64.4) | 93 (64.1) |
| Latino | | | |
| Unknown | 1 (1.4) | 0 | 1 (0.7) |
| Mean (SD) BMI, | 35.2 (5.7) | 33.5 (5.0) | 34.3 (5.4) |
| kg/m² | | | |
| Mean (SD) Body | 100.2 (18.9) | 94.4 (18.9) | 97.3 (19.1) |
| Weight, kg | | | |
| T2DM (stratification) | | | |
| Yes | 24 (33.3) | 24 (32.9) | 48 (33.1) |
| No | 48 (66.7) | 49 (67.1) | 97 (66.9) |
| With T2DM | | | |
| Mean (SD) A1C, % | 6.3 (0.7) | 6.5 (0.8) | 6.4 (0.7) |
| Mean (SD) FPG, | 121.9 (33.0) | 122.3 (31.0) | 122.1 (31.7) |
| mg/dL | | | |
| Without T2DM | | | |
| Mean (SD) A1C, % | 5.7 (0.3) | 5.6 (0.4) | 5.6 (0.4) |
| Mean (SD) FPG, | 101.6 (13.0) | 99.1 (11.4) | 100.3 (12.2) |
| mg/dL | | | |
| Mean (SD) LFC, % | 21.1 (8.1) | 19.4 (8.1) | 20.3 (8.1) |

Among 145 randomized subjects (efinopegdutide, N=72; semaglutide, N=73), the majority (55.2%) of participants were male, mean age was 49.5 years, mean body mass index was 34.3 kg/m², mean body weight was 97.3 kg, 33.1% of participants had T1DM, and mean LFC was 20.30.

### Efficacy

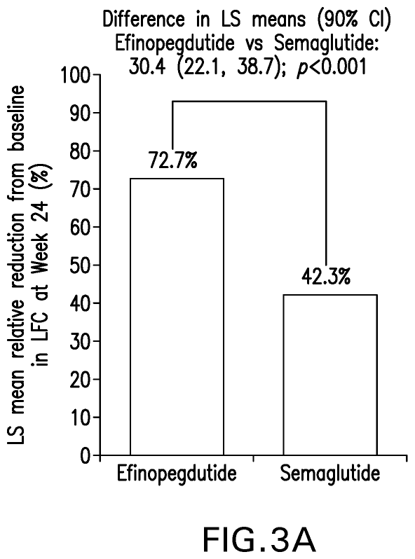

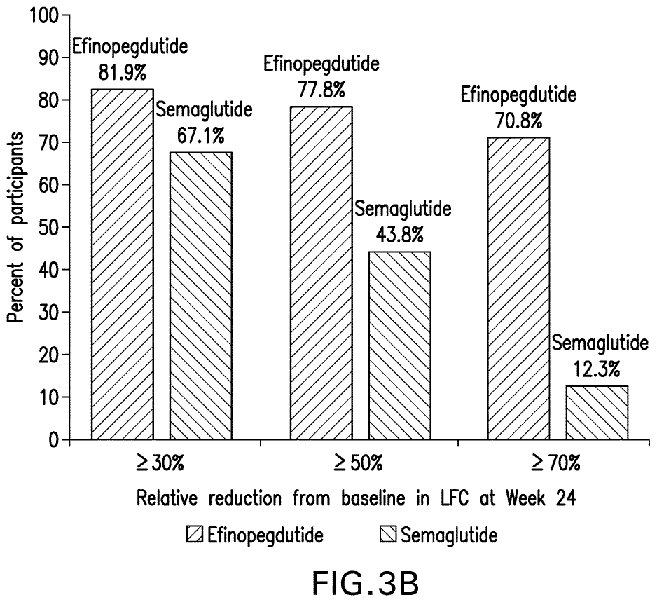

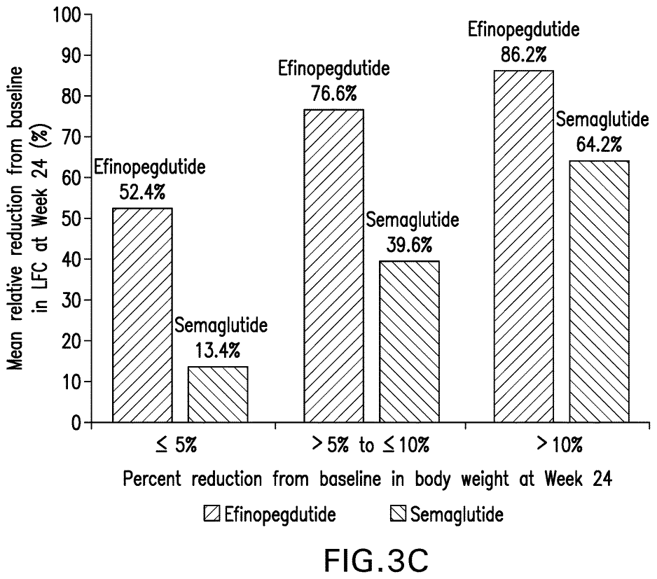

At Week 24, treatment with efinopegdutide led to a significantly (p<0.001) greater relative reduction from baseline in LFC compared to semaglutide; the LS mean relative reduction from baseline in LFC was 72.7% (90% Cl: 66.8, 78.7) with efinopegdutide and 42.3% (36.5, 48.1) with semaglutide (FIG. 3A, Table 2).

TABLE 2
Efficacy results

| | | | | | Percent Change from Baseline at Week 24 | | | |
|---|---|---|---|---|---|---|---|---|
| | | | | | | | | Difference |
| | | | | | | | | in LS |
| | | | | | | | | means |
| | | | | | | | | (90% CI) |
| | | Baseline | | Week 24 | | | | Efino vs. |
| | | mean | | mean | | | LS mean | Sema^{†} |
| Parameter | N | (SD) | N | (SD) | N | Mean (SD) | (90% CI) | p-value^{‡} |
| LFC, % | | | | | | | | |
| Efino | 72 | 21.1 (8.1) | 65 | 4.6 (4.5) | 72 | −74.6 (28.0) | −72.7 (−66.8, −78.7) | −30.4 (−22.1, −38.7) |
| Sema | 73 | 19.4 (8.1) | 72 | 11.4 (7.2) | 73 | −40.9 (27.9) | −42.3 (−36.5, −48.1) | <0.001 |
| Body | | | | | | | | −1.4 (−2.7, |
| weight, | | | | | | | | −0.1) |
| kg | | | | | | | | |
| Efino | 72 | 100.2 (18.8) | 66 | 91.6 (19.5) | 72 | −8.7 (5.2) | −8.5 (−9.5, −7.5) | 0.085 |
| Sema | 73 | 94.5 (18.9) | 71 | 87.4 (19.0) | 73 | −7.5 (4.3) | −7.1 (−8.1, −6.2) | |
| Total | | | | | | | | −7.2 (−11.2, |
| cholesterol, | | | | | | | | −3.1) |
| mmol/L | | | | | | | | |
| Efino | 72 | 5.3 (1.1) | 65 | 4.4 (1.0) | 72 | −16.4 (14.8) | −15.2 (−18.2, −12.2) | |
| Sema | 73 | 5.3 (1.2) | 69 | 4.7 (1.0) | 73 | −8.6 (15.3) | −8.0 (−11.0, −5.0) | |
| High- | | | | | | | | −11.7 (−15.8, |
| density | | | | | | | | −7.7) |
| lipoprotein | | | | | | | | |
| cholesterol, | | | | | | | | |
| mmol/L | | | | | | | | |
| Efino | 72 | 1.2 (0.3) | 65 | 1.1 (0.3) | 72 | −8.5 (15.3) | −8.1 (−11.2, −5.1) | |
| Sema | 73 | 1.2 (0.4) | 68 | 1.2 (0.3) | 73 | 2.0 (15.0) | 3.6 (0.6, 6.6) | |
| Low- | | | | | | | | −6.1 (−12.0, |
| density | | | | | | | | −0.1) |
| lipoprotein | | | | | | | | |
| cholesterol, | | | | | | | | |
| mmol/L | | | | | | | | |
| Efino | 71 | 3.3 (0.9) | 65 | 2.7 (0.8) | 72 | −14.9 (19.5) | −13.0 (−17.4, −8.6) | |
| Sema | 73 | 3.2 (1.1) | 67 | 2.8 (0.9) | 73 | −8.3 (22.9) | −6.9 (−11.3, −2.6) | |
| Triglycerides, | | | | | | | | −7.6 (−14.3, |
| mmol/L | | | | | | | | −0.9) |
| Efino | 72 | 2.0 (0.9) | 65 | 1.3 (0.5) | 72 | −26.1 (26.0) | −30.9 (−35.6, −25.8) | |
| Sema | 73 | 2.0 (1.0) | 68 | 1.5 (0.6) | 73 | −18.6 (27.3) | −23.3 (−28.5, −17.7) | |

Efino = efinopegdutide
Sema = semaglutide
For baseline and Week 24, N is the number of participants with non-missing assessments at the specific timepoint. For percent change from baseline, N is the number of randomized participants.
^{†}Based on a constrained longitudinal data analysis model including terms for treatment, time, stratum (T2DM), and the interactions of treatment by stratum, time by treatment, and time by stratum.
^{‡}p-value not computed for total cholesterol, high-density lipoprotein cholesterol, low-density lipoprotein cholesterol, and triglycerides.
LFC = liver fat content, CI = confidence interval, LS = least squares, SD = Standard Deviation.

Median relative reductions from baseline in LFC at Week 24 were 83.80% with efinopegdutide and 44.40% with semaglutide. A greater proportion of participants achieved a normal LFC level (less than 5%) at Week 24 with efinopegdutide (66.7% c) compared with semaglutide (17.8%). Greater proportions of participants had relative reductions from baseline in LFC at Week 24 of 30% or greater, 50% or greater, and 70% or greater with efinopegdutide (81.9%, 77.8%, and 70.80%, respectively) compared with semaglutide (67.10%, 43.8%, and 12.30%, respectively) (FIG. 3B).

Both treatment groups had an LS mean percent reduction from baseline in body weight at Week 24 (efinopegdutide 8.500 vs semaglutide 7.100; p=0.085) (Table 2).

The relative reductions from baseline in LFC at Week 24 by weight-loss category (5% or less, greater than 50% to 10% or less, and greater than 10% reduction in body weight from baseline) were greater in the efinopegdutide group (52.4%, 76.6%0, and 86.2%, respectively) than in the semaglutide group (13.4%, 39.6%, and 64.2%, respectively) (FIG. 3Q).

In the efinopegdutide group, LS mean percent reductions from baseline were observed in total cholesterol, high-density lipoprotein cholesterol, low-density lipoprotein cholesterol, and triglycerides of 15.2%, 8.1%, 13.0%, and 30.9%, respectively, at Week 24 (Table 2). In the semaglutide group, LS mean percent reductions from baseline in total cholesterol, low-density lipoprotein cholesterol, and triglycerides of 8.0%, 6.9%, and 23.3%, respectively, and an LS mean percent increase from baseline in high-density lipoprotein cholesterol of 3.6%, were observed at Week 24 (Table 2).

### Safety

Slightly higher incidences of adverse events and drug-related adverse events were observed in the efinopegdutide group, primarily related to an imbalance in gastrointestinal adverse events (Table 3).

TABLE 3
Adverse Events

| | Efinopegdutide | | Semaglutide | |
|---|---|---|---|---|
| | N = 72 | | N = 73 | |
| | n | (%) | n | (%) |
| Adverse events | 64 | (88.9) | 53 | (72.6) |
| Drug-related† adverse events | 46 | (63.9) | 35 | (47.9) |
| Serious adverse events | 1 | (1.4) | 1 | (1.4) |
| Serious drug-related adverse events | 0 | | 0 | |
| Deaths | 0 | 0 | 0 | |
| Discontinued drugdue to an adverse event | 4 | (5.6) | 0 | |
| Discontinued drug due to a drug-related | 3 | (4.2) | 0 | |
| adverse event | | | | |
| Discontinued drug due to a serious | 0 | | 0 | |
| adverse event | | | | |
| Discontinued drug due to a serious | 0 | | 0 | |
| drug-related adverse event | | | | |
| Adverse events that occurred in ≥4 | | | | |
| participants in either treatment group | | | | |
| Gastrointestinal adverse events | | | | |
| Abdominal distension | 4 | (5.6) | 3 | (4.1) |
| Abdominal pain | 9 | (12.5) | 2 | (2.7) |
| Abdominal pain upper | 7 | (9.7) | 1 | (1.4) |
| Constipation | 12 | (16.7) | 4 | (5.5) |
| Diarrhea | 12 | (16.7) | 13 | (17.8) |
| Dyspepsia | 6 | (8.3) | 5 | (6.8) |
| Flatulence | 4 | (5.6) | 1 | (1.4) |
| Gastroesophageal reflux disease | 6 | (8.3) | 5 | (6.8) |
| Nausea | 20 | (27.8) | 23 | (31.5) |
| Vomiting | 12 | (16.7) | 11 | (15.1) |
| General disorders and | | | | |
| administration site conditions | | | | |
| Fatigue | 1 | (1.4) | 6 | (8.2) |
| Infections and infestations | | | | |
| COVID-19 | 8 | (11.1) | 10 | (13.7) |
| Urinary tract infection | 4 | (5.6) | 2 | (2.7) |
| Investigations | | | | |
| Alanine aminotransferase increased | 4 | (5.6) | 0 | |
| Lipase increased | 4 | (5.6) | 3 | (4.1) |
| Metabolism and nutrition disorders | | | | |
| Decrease appetite | 12 | (16.7) | 11 | (15.1) |
| Nervous system disorders | | | | |
| Dizziness | 4 | (5.6) | 2 | (2.7) |
| Headache | 5 | (6.9) | 5 | (6.8) |

†Considered by the investigator to be related to the drug.

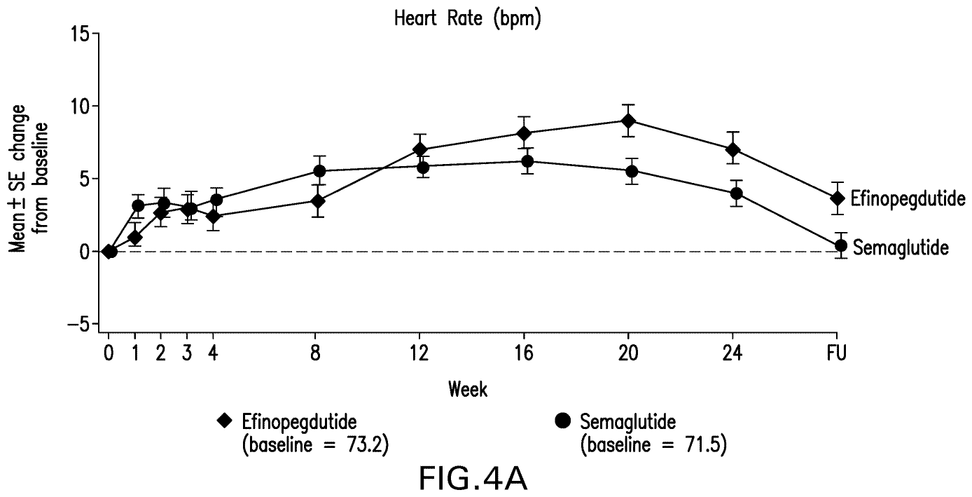

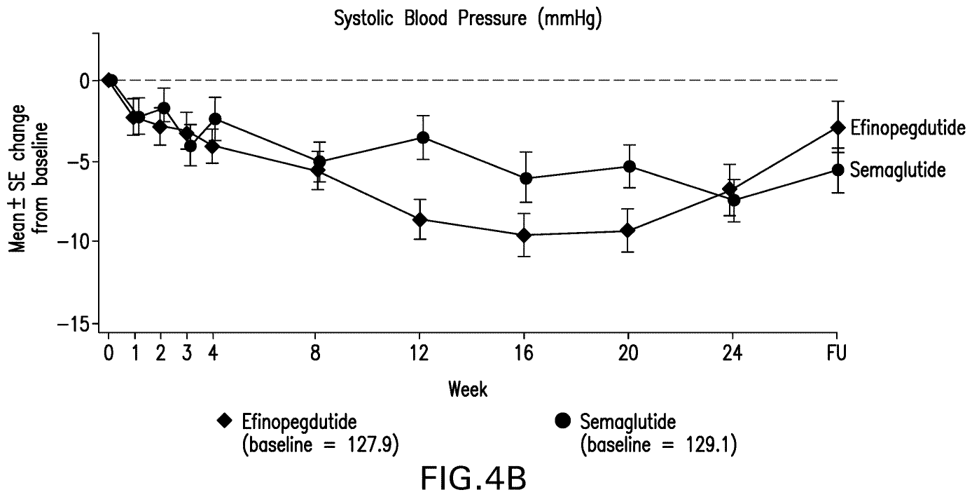

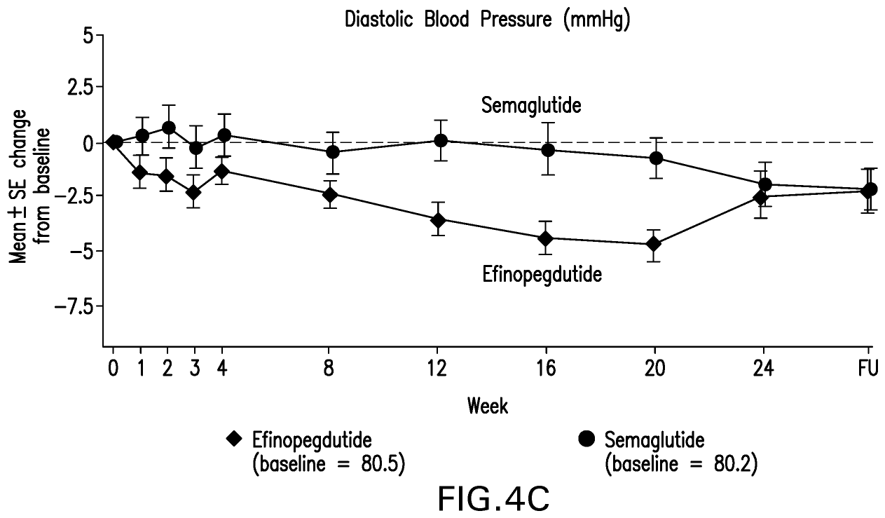

There were otherwise no meaningful differences between the two treatment groups in the incidence of overall, serious, or drug-related adverse events, including adverse events that led to discontinuation (Table 3). The overall profile of adverse events reported by at least 4 participants was similar between the two treatment groups (Table 3). The incidences of nausea and of vomiting were similar between the two treatment groups (Table 3). Three specific adverse events in the Gastrointestinal System Organ Class were reported at a higher incidence with efinopegdutide compared to semaglutide: abdominal pain (12.5% vs. 2.7%, respectively), abdominal pain upper (9.7% vs. 1.4%), and constipation (16.7% vs. 5.5%) (Table 3). Slightly greater increases from baseline in mean heart rate, and slightly greater reductions from baseline in both mean systolic and mean diastolic blood pressure, were observed with efinopegdutide compared to semaglutide (FIGS. 4A-4C). There was no notable imbalance in adverse events considered potentially related to changes in heart rate or blood pressure.

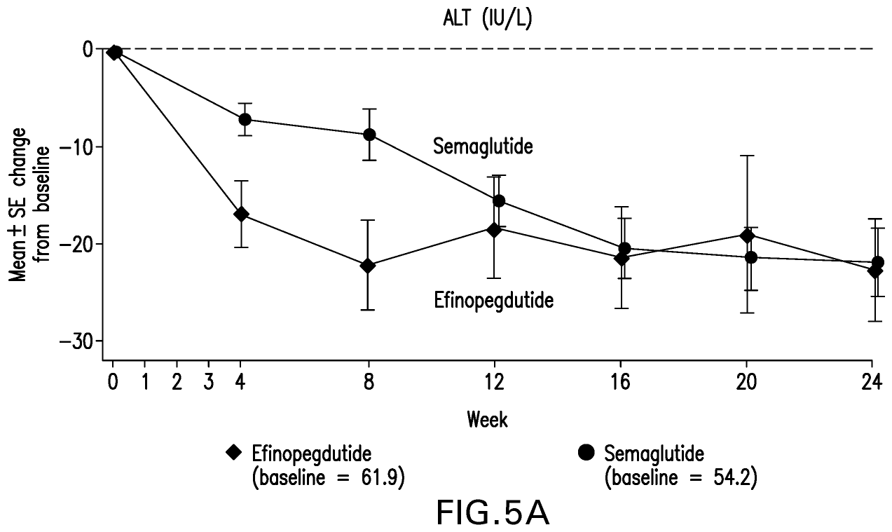

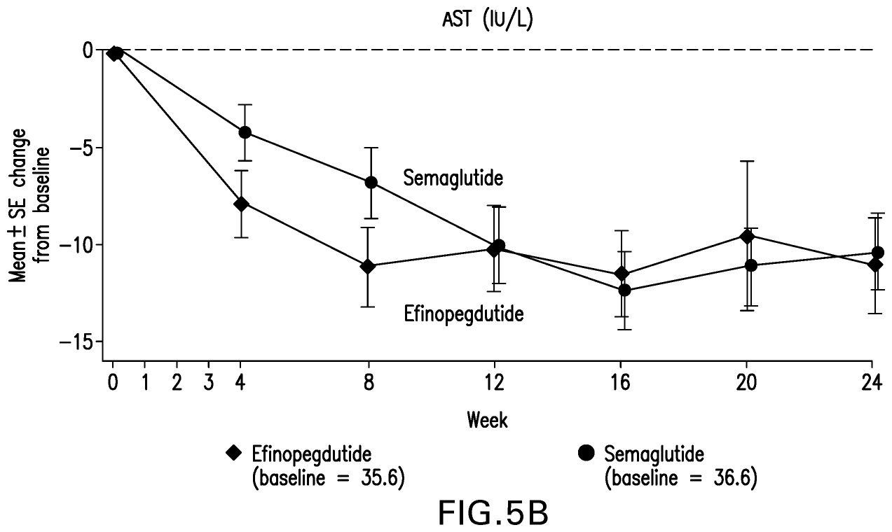

While there was a small imbalance in the number of subjects with the adverse event of alanine aminotransferase (ALT) increased, similar reductions from baseline in mean ALT and mean aspartate aminotransferase (AST) were observed in the two treatment groups, which had begun by Week 4 and continued throughout the 24-week treatment period (FIGS. 5A-5B). Treatment with efinopegdutide was associated with a mean increase from baseline in fasting plasma glucose of 0.2 mg/dL and a mean change from baseline in A1C of 0.0% at Week 24, compared to a mean decrease in fasting plasma glucose of 11.6 mg/dL and a mean decrease in AlC of 0.5% with semaglutide. Treatment with efinopegdutide and semaglutide was associated with a mean decrease from baseline in hemoglobin of 1.0 g/dL and 0.2 g/dL, respectively, at Week 24. There were no adverse events related to reductions in hemoglobin in either treatment group. There were no meaningful differences for the other laboratory assessments.

TABLE 4
Amino acid sequences

| SEQ | | |
|---|---|---|
| ID | | |
| NO: | Description | Sequence |
| 1 | Oxyntomodulin | HSQGTFTSDYSKYLDSRRAQDFVQWLMNTKRNRNNIA |
| | (OXM) | |
| 2 | OXM derivative of | HXQGTFTSDYSKYLDEKRAKEFVQWLMNTC |
| | Efinopegdutide | |
| | X2 is 2- | |
| | aminoisobutyric | |
| | acid (aib) | |
| | Features: | |
| | Lactam bridge | |
| | between E16 and | |
| | K20 | |
| | C-terminal amide | |
| 3 | IgG4 Fc: del1-8 of | PSCPAPEFLGGPSVFLFPPKPKDTLMISRTPEVTCVVVDVSQE |
| | hinge, CH2, and | DPEVQFNWYVDGVEVHNAKTKPREEQFNSTYRVVSVLTVL |
| | CH3 | HQDWLNGKEYKCKVSNKGLPSSIEKTISKAKGQPREPQVYT |
| | | LPPSQEEMTKNQVSLTCLVKGFYPSDIAVEWESNGQPENNY |
| | | KTTPPVLDSDGSFFLYSRLTVDKSRWQEGNVFSCSVMHEAL |
| | | HNHYTQKSLSLSLGK |
| 4 | GLP-1 derivative | HXEGTFTSDVSSYLEGQAALEFIAWLVRGRG |
| | of Semaglutide | |
| | X2 is aib | |
| | Features: | |
| | C-terminal | |
| | hydroxyl | |
| 5 | IgG4 Fc: del1-8 of | PSCPAPEFLGGPSVFLFPPKPKDTLMISRTPEVTCVVVDVSQE |
| | hinge, CH2, and | DPEVQFNWYVDGVEVHNAKTKPREEQFNSTYRVVSVLTVL |
| | CH3, del C- | HQDWLNGKEYKCKVSNKGLPSSIEKTISKAKGQPREPQVYT |
| | terminal K | LPPSQEEMTKNQVSLTCLVKGFYPSDIAVEWESNGQPENNY |
| | | KTTPPVLDSDGSFFLYSRLTVDKSRWQEGNVFSCSVMHEAL |
| | | HNHYTQKSLSLSLG |

### REFERENCES

Younossi Z M, Koenig A B, Abdelatif D, Fazel Y, Henry L, Wymer M. Global epidemiology of nonalcoholic fatty liver disease-meta-analytic assessment of prevalence, incidence, and outcomes. Hepatology 2016; 64:73-84.
Kechagias S, Nasr P, Blomdahl J, Ekstedt M. Established and emerging factors affecting the progression of nonalcoholic fath, liver disease. Metabolism 2020:111:154183.
Sheka A C, Adeyi 0, Thompson J, Hameed B, Crawford P A, Ikramuddin S. Nonalcoholic steatohepatitis: a review. JAMA 2020; 323:1175-1183. Erratum in: JAMA 2020:323:1619.
Fabbrini E, Sullivan S, Klein S. Obesity and nonalcoholic fatty liver disease: biochemical, metabolic, and clinical implications. Hepatology 2010; 51:679-689.
Godoy-Matos A F, Silva Jnnior W S, Valerio C M. NAFLD as a continuum: from obesity to metabolic syndrome and diabetes. Diabetology & Metabolic Syndrome 2020; 12:60.
Rinella M E, Neuschwander-Tetri B A, Siddiqui M S, Abdelmalek M F, Caldwell S, et al. AASLD practice guidance on the clinical assessment and management of nonalcoholic fatty liver disease. Hepatology 2023; 10.1097/HEP.0000000000000323.
SAXENDA® (liraglutide) injection, U.S. package insert. Available at: www.accessdata.fda.gov/drugsatfda_docs/label/2018/206321s0071bl.pdf. Last accessed 23 Feb. 2023.
WEGOVY® (semaglutide) injection, U.S. package insert. Available at: www.accessdata.fda.gov/drugsatfda_docs/label/2022/215256s0051bl.pdf. Last accessed 23 Feb. 2023.
Armstrong M J, Gaunt P, Aithal G P, Barton D, Hull D. Parker R, et al. Liraglutide safety and efficacy in patients with non-alcoholic steatohepatitis (LEAN): a multicentre, doubleblind, randomised, placebo-controlled phase 2 study. Lancet 2016:387:679-690.
Newsome P N, Buchholtz K, Cusi K, Linder M, Okanoue T, Ratziu V, et al. A placebo-controlled trial of subcutaneous semaglutide in nonalcoholic steatohepatitis. N Engl J Med 2021; 384:1113-1124.
Seghieri M. Christensen A S, Andersen A, Solini A, Knop F K. Vilsboll T. Future perspectives on GLP-1 receptor agonists and GLP-1/glucagon receptor co-agonists in the treatment of NAFLD. Front Endocrinol 2018; 9:649.
Boland M L, Laker R C, Mather K, Nawrocki A, Oldham S, Boland B B, et al. Resolution of NASH and hepatic fibrosis by the GLP-1R and GCGR dual-agonist cotadutide via modulating mitochondrial function and lipogenesis. Nat Metab 2020; 2:413-431.
Wynne K, Park A J, Small C J, Meeran K, Ghatei M A, Frost G S, et al. Oxyntomodulin increases energy expenditure in addition to decreasing energy intake in overweight and obese humans: A randomised controlled trial. Int J Obes 2006; 30:1729-1136.
Wynne K, Park A J, Small C J, Patterson M, Ellis S M, Murphy K G, et al. Subcutaneous oxyntomodulin reduces body weight in overweight and obese subjects: a double-blind, randomized, controlled trial. Diabetes 2005; 54:2390-2395.
Alba M, Yee J. Frustaci M E. Samtani M N. Fleck P. Efficacy and safety of glucagon-like peptide-1/glucagon receptor co-agonist JNJ-64565111 in individuals with obesity without type 2 diabetes mellitus: A randomized dose-ranging study. Clin Obes 2021; 11:e12432. doi:10.1111/cob.12432
Di Prospero N A, Yee J. Frustaci M E, Samtani M N, Alba M. Fleck P. Efficacy and safety of glucagon-like peptide-1/glucagon receptor co-agonist JNJ-64565111 in individuals with type 2 diabetes mellitus and obesity: A randomized dose-ranging study. Clin Obes 2021; 11:e12433. doi:10.1111/cob.12433.
Liang K-Y, Zeger S L. Longitudinal data analysis of continuous and discrete responses for pre-post designs. The Indian J Stat 2000; 62(Series B, Pt. 1):134-148.
Flint A, Andersen G, Hockings P, Johansson L, Morsing A, Palle M S, et al. Randomised clinical trial: semaglutide versus placebo reduced liver steatosis but not liver stiffness in subjects with non-alcoholic fatty liver disease assessed by magnetic resonance imaging. Aliment Pharmacol Ther 2021; 54:1150-1161.
Alkhouri N, Herring R, Kabler H, Kayali Z, Hassanein T, Kohli A, et al. Safety and efficacy of combination therapy with semaglutide, cilofexor and firsocostat in patients with non-alcoholic steatohepatitis: A randomised, open-label phase II trial. J Hepatol 2022; 77:607-618.
Loomba R, Abdelmalek M F, Armstrong M, Jara M, Kjaer M, Krarup N, et al. Semaglutide 2.4 mg once weekly improved liver and metabolic parameters, and was well tolerated, in patients with nonalcoholic steatohepatitis-related cirrhosis: a randomised, placebo-controlled phase 2 trial. J Hepatol 2022; 77 (suppl. 1):S10.
Promrat K, Kleiner D E, Niemeier H M. Jackvony E, Kearns M, Wands J R, et al. Randomized controlled trial testing the effects of weight loss on nonalcoholic steatohepatitis. Hepatology 2010; 51:121-129.
Petit J-M, Cercueil J-P, Loffroy R, Denimal D, Bouillet B, Fourmont C, et al. Effect of liraglutide therapy on liver fat content in patients with inadequately controlled type 2 diabetes: the LIRA-NAFLD study. J Clin Endocrinol Metab 2017; 102:407-415.
Mattar S G, Velcu L M, Rabinovitz M. Demetris A J, Krasinskas A M, Barinas-Mitchell E, et al. Surgically-induced weight loss significantly improves nonalcoholic fatty liver disease and the metabolic syndrome. Ann Surg 2005; 242:610-620.
Gluszyńska P, Lemancewicz D, Dzieciol J B. Hady H R. Non-alcoholic fatty liver disease (NAFLD) and bariatric/metabolic surgery as its treatment option: A review. J Clin Med 2021:10:5721.
De Block C E M, Dirinck E. Verhaegen A, Van Gaal L F. Efficacy and safety of high-dose glucagon-like peptide-1, glucagon-like peptide-1/glucose-dependent insulinotropic peptide, and glucagon-like peptide-1/glucagon receptor agonists in type 2 diabetes. Diabetes Obes Metab 2022; 24:788-805.
Naets J P, Guns M. Inhibitory effect of glucagon on ervthropoiesis. Bloo. 1980; 55:997-1002.
Yang W, Yan H, Pan Q, Shen J Z, Zhou F, Wu C, et al. Glucagon regulates hepatic mitochondrial function and biogenesis through Foxol, J Endocrinol. 2019; 241:265-278.
Soga J, Yakuwa Y. Glucagonomas/diabetico-dermatogenic syndrome (DDS): A statistical evaluation of 407 reported cases. J Hepatobiliary Pancreat Surg 1998; 5:312-319.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for reducing liver fat content (LFC) in an individual with fatty liver disease, comprising:
administering a dose from about 2 mg to about 10 mg of efinopegdutide or a pharmaceutically acceptable salt thereof to the individual once weekly for a time sufficient to reduce the LFC in the individual.

2. The method of claim 1, where the dose is about 2 mg of efinopegdutide or the pharmaceutically acceptable salt thereof.

3. (canceled)

4. The method of claim 1, where the dose is about 4 mg of efinopegdutide or the pharmaceutically acceptable salt thereof.

5. (canceled)

6. The method of claim 1, where the dose is about 7 mg of efinopegdutide or the pharmaceutically acceptable salt thereof.

7. (canceled)

8. The method of claim 1, where the dose is about 10 mg of efinopegdutide or the pharmaceutically acceptable salt thereof.

9. (canceled)

10. The method of claim 1, wherein the fatty liver disease is nonalcoholic fatty liver disease (NAFLD) or nonalcoholic steatohepatitis (NASH).

11. The method of claim 10, wherein the NASH is pre-cirrhotic NASH or cirrhotic NASH.

12-13. (canceled)

14. The method of claim 1, wherein the individual with fatty liver disease has an LFC of 5% or greater prior to administering the efinopegdutide or pharmaceutically acceptable salt thereof.

15-18. (canceled)

19. The method of claim 1, wherein the individual has type 1 diabetes mellitus (T1DM) or type 2 diabetes mellitus (T2M).

20. The method of claim 19, wherein the individual has type 2 diabetes mellitus (T2M) and is overweight or obese.

21. The method of claim 1, wherein the individual is overweight or obese.

22. A method for treating fatty liver disease in an individual in need thereof, comprising:
administering a dose from about 2 mg to about 10 mg of efinopegdutide or a pharmaceutically acceptable salt thereof to the individual once weekly for a time sufficient to treat the fatty liver disease in the individual.

23. The method of claim 22, where the dose is about 2 mg of efinopegdutide or the pharmaceutically acceptable salt thereof.

24. (canceled)

25. The method of claim 22, where the dose is about 4 mg of efinopegdutide or the pharmaceutically acceptable salt thereof.

26. (canceled)

27. The method of claim 22, where the dose is about 7 mg of efinopegdutide or the pharmaceutically acceptable salt thereof.

28. (canceled)

29. The method of claim 22, where the dose is about 10 mg of efinopegdutide or the pharmaceutically acceptable salt thereof.

30. (canceled)

31. The method of claim 22, wherein the fatty liver disease is nonalcoholic fatty liver disease (NAFLD) or nonalcoholic steatohepatitis (NASH).

32. The method of claim 31, wherein the NASH is pre-cirrhotic NASH or cirrhotic NASH.

33-42. (canceled)

43. A method for reducing liver fat content (LFC) to less than 5% in an individual with elevated LFC comprising in the following order the steps of:
(a) administering to the individual a dose of about 2.0 mg of efinopegdutide or a pharmaceutically acceptable salt thereof once a week for four weeks,
(b) administering to the individual a dose of about 5.0 mg of efinopegdutide or a pharmaceutically acceptable salt thereof once a week for four weeks, and
(c) administering to the individual a dose of about 10 mg of efinopegdutide or a pharmaceutically acceptable salt thereof once a week for a time sufficient to reduce the LFC to less than 5%.

44-62. (canceled)

63. A method for treating fatty liver disease in an individual in need thereof comprising in the following order the steps of:
(a) administering to the individual a dose of about 2.0 mg of efinopegdutide or a pharmaceutically acceptable salt thereof once a week for four weeks,
(b) administering to the individual a dose of about 5.0 mg of efinopegdutide or a pharmaceutically acceptable salt thereof once a week for four weeks, and
(c) administering to the individual a dose of about 10 mg of efinopegdutide or a pharmaceutically acceptable salt thereof once a week for a time sufficient to treat the fatty liver disease in the individual.

64-125. (canceled)


SEQUENCE LISTING (5 sequences)

SEQ ID NO: 1 - protein, 37 aa, unidentified
HSQGTFTSDYSKYLDSRRAQDFVQWLMNTKRNRNNIA

SEQ ID NO: 2 - protein, 30 aa, synthetic construct
  SITE 2: note = Aib
  REGION 16..20: note = residues linked by lactam bridge
  SITE 30: note = Amidated residue
HXQGTFTSDYSKYLDEKRAKEFVQWLMNTC

SEQ ID NO: 3 - protein, 221 aa, synthetic construct
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 31 aa, synthetic construct
  SITE 2: note = Aib
  SITE 31: note = Hydroxylated residue
HXEGTFTSDVSSYLEGQAALEFIAWLVRGRG

SEQ ID NO: 5 - protein, 220 aa, synthetic construct
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG